(19) United States
(12) Patent Application Publication
(10) Pub. No.: US 2023/0218644 A1
(43) Pub. Date: Jul. 13, 2023

(54) COMPOUNDS FOR USE IN THE TREATMENT OF VIRAL INFECTIONS BY RESPIRATORY SYNDROME-RELATED CORONAVIRUS

(71) Applicants: SOM INNOVATION BIOTECH, S.A., Barcelona (ES); EWHA WOMANS UNIVERSITY, Seoul (KR)

(72) Inventors: Raúl INSA BORONAT, Barcelona (ES); Luca SIGNORILE, Barcelona (ES); Núria REIG BOLAÑO, Barcelona (ES); Dong-Hae SHIN, Seodaemun-gu, Seoul (KR)

(21) Appl. No.: 17/996,362

(22) PCT Filed: Apr. 15, 2021
(86) PCT No.: PCT/EP2021/059808

§ 371 Date: Oct. 17, 2022

(30) Foreign Application Priority Data
Apr. 16, 2020 (EP) 20382303.4

(51) Int. Cl. A61K 31/65; A61P 31/14; A61K 31/216; A61K 31/497

(52) U.S. Cl. CPC A61K 31/65; A61P 31/14; A61K 31/216; A61K 31/497

(57) ABSTRACT

The present invention relates to a compound selected from the group consisting of cynarine, eravacycline and prexasertib or a pharmaceutically acceptable salt thereof, preferably eravacycline or a pharmaceutically acceptable salt thereof, and to combination of said compounds with other active ingredients, for use in the treatment and/or prevention of viral infections by coronavirus selected from the group consisting of MERS-CoV, SARS-CoV and SARS-CoV-2 virus, to the use of said compound or its combinations in the manufacture of a medicament for the treatment or prevention of said diseases and to a method of treating and/or preventing by administration of said compound or its combinations.

### FIELD OF THE INVENTION

The present invention relates to compounds for use in the treatment and/or prevention of viral infections by coronavirus, especially by respiratory syndrome-related coronavirus selected from the species “Middle East respiratory syndrome-related coronavirus” (such as MERS-CoV) and “Severe respiratory syndrome-related coronavirus” (such as SARS-CoV and SARS-CoV-2).

### BAKGROUND OF THE INVENTION

The respiratory syndrome-related coronavirus are a group of related betacoronaviruses, a genus the subfamily Orthocoronavirinae, in the family Coronaviridae, order Nidovirales, and realm Riboviria.

They are enveloped viruses with a positive-sense single-stranded RNA genome and a nucleocapsid of helical symmetry. The name coronavirus is derived from the Latin corona, meaning “crown” or “halo”, which refers to the characteristic appearance reminiscent of a solar corona around the virions (virus particles) when viewed under two-dimensional transmission electron microscopy, due to the surface being covered in club-shaped protein spikes.

The overall structure of betacoronavirus genome is similar to that of other Coronaviruses, with an ORF1ab replicase polyprotein (rep, pp1ab) preceding other elements. This polyprotein is cleaved into many nonstructural proteins (including a 3C-like protease −3CLpro).

In December 2019 a new infectious disease caused by severe acute respiratory syndrome coronavirus 2 (SARS-CoV-2) was first identified in Wuhan, the capital of China's Hubei province, and has since spread globally, resulting in the ongoing 2019-20 coronavirus pandemic. The disease has been given the name Coronavirus disease 2019 or COVID-19.

Common symptoms include fever, cough, and shortness of breath. Other symptoms may include muscle pain, sputum production, diarrhea, sore throat, loss of smell, and abdominal pain. While the majority of cases result in mild symptoms, some progress to bilateral pneumonia and multi-organ failure.

SARS-CoV-2 is a positive-sense single-stranded RNA virus with approximately 29,900 nucleotides. Similar to the genome of other betacoronaviruses, SARS-CoV-2's genome contains two flanking untranslated regions (UTRs) and a single long open reading frame encoding a polyprotein. The 5′-terminal two-thirds of the genome encodes a polyprotein, pp1ab, which is further cleaved into 16 nonstructural proteins that are involved in genome transcription and replication. The 3′ terminus encodes structural proteins, including envelope glycoproteins spike (S), envelope (E), membrane (M) and nucleocapsid (N).

After SARS-CoV-2 infection, the open reading frame, ORF1ab, of the viral genome is translated into a polyprotein (the orf1ab polyprotein having 7096 aminoacids), from which sixteen non-structural proteins (Nsp1-Nsp16), including the RNA-dependent RNA polymerase (RdRp, Nsp12), helicase (Nsp13), papain-like protease (Nsp3) and 3C-like proteinase (3CLpro, main protease or Mpro, Nsp5), among other Nsps involved in the transcription and replication of the virus, are released by extensive proteolytic processing. This is primarily achieved by the 306-aminoacid 3C-like protease (3CLpro), a Cys protease. The functional importance of 3CLpro in the viral life cycle, together with the absence of closely related homologues in humans, makes the 3CLpro an attractive target for the development of drugs directed against SARS and other coronavirus infections.

3CLPro cleaves polyproteins within the Leu-Gln↓(Ser, Ala, Gly) sequence (↓ indicates the cleavage site), which appears to be a conserved pattern of this protease. The ability of peptide bond hydrolysis after Gln residues is also observed for main proteases of other coronaviruses (3CLPro of SARS-CoV-2 and SARS-CoV differ by only 12 amino acids with a homology of greater than 96%, and the structures of the two are basically the same) but is unknown for human enzymes. This observation, along with further studies on the 3CLPro can potentially lead to new broad-spectrum anti-coronaviral inhibitors with minimum side effects.

In addition, the protein may cleave the intracellular protein NEMO and thereby inhibit the activation of the interferon signaling pathway. Therefore, inhibition of 3CLPro can effectively inhibit virus infection and replication.

The importance of the main protease as a target for antiviral drugs is shared with other viruses, for example selective inhibitors of the Human Immunodeficiency Virus (HIV) protease are effective at preventing HIV replication and are successfully used for the treatment of AIDS (acquired immune deficiency syndrome) as single drugs or in combination with other antiviral agents. Similarly, selective inhibitors of the Hepatitis C Virus (HCV) NS3/4A protease are successfully used for the treatment of HCV infections.

Since the epidemic, many articles based on the 3CLPro structure have virtually screened drugs that may be used for the treatment of new coronavirus pneumonia through computer simulations.

Chen et al. screened out the drugs that have been marketed through structural simulation (https://f1000research.com/articles/9-129/v1/doi: f1000research.com/articles/9-129/v1).

Qamar et al. Screened 32297 potential antiviral medicinal plant compounds based on 3CLPro and found 9 that could be optimized for anti-COVID-19 therapeutic effects (https://reader.elsevier.com/reader/sd/pii/S2095177920301271?token=B7529755FDF4F82829C337E5A98949890886620A3F5FA2AD9D287F200EDA5FDA8FCC381C84BD6C9764D2D58FB38567A2/doi:10.1016/j.jpha.2020.03.009).

Alessandro Contini identified 3 potential drugs, including Indinavir, Lopinavir and Atazanavir and Cobicistat (origin target: CYP3A), through virtual screening from 3118 FDA-approved drugs (https://chemrxiv.org/articles/Virtual_Screening_of_an_FDA_Approved_Drugs_Database_on_Two_COVID-19_Coronavirus_Proteins/11847381/1/doi:10.26434/chemrxiv.11847381.v1)).

Jared S. Morse et al. suggested that compound 3CLPro-1 can inhibit SARS and MERS virus 3CLPro activity at nM level and may be effective for SARS-CoV-2 3CLPro (https://chemistry-europe.onlinelibrary.wiley.com/doi/full/10.1002/cbic.202000047; doi: 10.1002/cbic.202000047)

Wang J. et al, J. Chem. Inf. Model. V. 60, pp 3277-3286, 2020 describes the use of docking and molecular dynamics technologies as a tool for the selection of compounds with potential SARS-CoV-2 3CLpro inhibition properties. The article identifies several compounds with potential activity: carfilzomib, eravacycline, valrubicin, lopinavir, and elbasvir, in addition to several bioactive compounds with similar structure to lopinavir. However, the article fails to provide any data on the capacity of said products to actually inhibit SARS-CoV-2 3CLpro. In fact, another paper published a few month later (Fu et al., Nature Comm., V. 11, No. 4417, 2020) showed that carfilzomib, one of potential binding agents identified in Wang, failed to inhibit SARS-CoV-2 protease.

A few publications went beyond virtual screening and reported either the results of testing the capacity of several compounds to inhibit has SARS-CoV-2 3CLPro protease or to inhibit viral infection with SARS-CoV-2 of Vero E6 cells.

Haixia Su et al., bioRxiv preprint doi: https://doi.org/10.1101/2020.04.13.038687; described that baicalin and baicalein were inhibitors of the 3CLpro protease of SARS-CoV-2 and were also effective in inhibiting infection of Vero E6 cells by the virus.

Zhijian Xu and others showed that nelfinavir has SARS-CoV-2 3CLPro inhibitory activity in vitro and suggested that may be effective as a COVID-19 treatment (https://chemrxiv.org/articles/Nelfinavir_Is_Active_Against_SARS-CoV-2_in_Vero_E6_Cells/12039888/1; doi: 10.26434/chemrxiv.12039888.v1). This drug, among many others, is currently in clinical trials for COVID-19.

Finally Choy K. et al, Antivir. Res. V. 178, pp 1-5, 2020 reported the antiviral effect of remdesivir, lopinavir, homorringtonine, and emetine against SARS-CoV-2 virus in Vero E6 cells with IC50 of 23.15 μM, 26.63 μM, 2.55 μM and 0.46 μM, respectively.

### DESCRIPTION OF THE FIGURES

FIG. 1 is a representation of the capacity of cynarine to inhibit SARS-CoV-2 3CL protease. Each data point represents the effect of the compound against SARS-CoV-2 3CLpro compared to the control. The effect is expresses as Relative Fluorescence Units plotted against the log-concentration of the compound.

FIG. 2 is a representation of the capacity of eravacycline to inhibit SARS-CoV-2 3CL protease. Each data point represents the effect of the compound against SARS-CoV-2 3CLpro compared to the control. The effect is expresses as Relative Fluorescence Units plotted against the log-concentration of the compound.

FIG. 3 is a representation of the capacity of prexasertib to inhibit SARS-CoV-2 3CL protease. Each data point represents the effect of the compound against SARS-CoV-2 3CLpro compared to the control. The effect is expresses as Relative Fluorescence Units plotted against the log-concentration of the compound.

FIG. 4 is a representation of the capacity of cynarine to inhibit MERS-CoV 3CL protease. Each data point represents the effect of the compound against MERS-CoV 3CLpro compared to the control. The effect is expresses as Relative Fluorescence Units plotted against the log-concentration of the compound.

FIG. 5 is a representation of the capacity of eravacycline to inhibit MERS-CoV 3CL protease. Each data point represents the effect of the compound against MERS-CoV 3CLpro compared to the control. The effect is expresses as Relative Fluorescence Units plotted against the log-concentration of the compound.

FIG. 6 is a representation of the capacity of prexasertib to inhibit MERS-CoV 3CL protease. Each data point represents the effect of the compound against MERS-CoV 3CLpro compared to the control. The effect is expresses as Relative Fluorescence Units plotted against the log-concentration of the compound.

FIG. 7 is a representation of the capacity of evaracycline to inhibit SARS-CoV-2 infection in VeroE6 cells. Circles represent inhibition of SARS-CoV-2 infection (%), and squares represent cell viability (%). Means SD were calculated from duplicate experiments. IC₅₀ and CC₅₀ values are indicated, as well as the Selectivity Index (SI: CC₅₀/IC₅₀).

FIG. 8 is a representation of the capacity of chloroquine to inhibit SARS-CoV-2 infection in VeroE6 cells. Circles represent inhibition of SARS-CoV-2 infection (%), and squares represent cell viability (%). Means SD were calculated from duplicate experiments. IC₅₀ and CC₅₀ values are indicated, as well as the Selectivity Index (SI: CC₅₀/IC₅₀).

FIG. 9 is a representation of the capacity of remdesivir to inhibit SARS-CoV-2 infection in VeroE6 cells. Circles represent inhibition of SARS-CoV-2 infection (%), and squares represent cell viability (%). Means SD were calculated from duplicate experiments. IC₅₀ and CC₅₀ values are indicated, as well as the Selectivity Index (SI: CC₅₀/IC₅₀).

### SUMMARY OF THE INVENTION

The inventors have surprisingly found new inhibitors of the 3CLPro protease of coronavirus such as MERS-CoV, SARS-CoV and specially SARS-CoV-2, which can be useful to arrest virus infection and replication.

Thus, in a first aspect, the present invention relates to a compound selected from the group consisting of cynarine, eravacycline and prexasertib or a pharmaceutically acceptable salt thereof, preferably eravacycline or a pharmaceutically acceptable salt thereof, for use in the treatment and/or prevention of viral infections by betacoronaviruses, especially by respiratory syndrome-related coronaviruses selected from the species “Middle East respiratory syndrome-related coronavirus” (MERS-CoV) and “Severe respiratory syndrome-related coronavirus” (SARS-CoV and SARS-CoV-2).

In a second aspect, the invention relates to the use of a compound selected from the group consisting of cynarine, eravacycline and prexasertib or a pharmaceutically acceptable salt thereof, preferably eravacycline or a pharmaceutically acceptable salt thereof, in the manufacture of a medicament for the treatment and/or prevention of viral infections by betacoronaviruses, especially by respiratory syndrome-related coronaviruses selected from the species “Middle East respiratory syndrome-related coronavirus” (such as MERS-CoV) and “Severe respiratory syndrome-related coronavirus” (such as SARS-CoV and SARS-CoV-2).

In a third aspect, the invention also relates to a method of treating and/or preventing viral infections by betacoronaviruses, especially by respiratory syndrome-related coronaviruses selected from the species “Middle East respiratory syndrome-related coronavirus” (such MERS-CoV) and “Severe respiratory syndrome-related coronavirus” (such as SARS-CoV and SARS-CoV-2), in a subject, comprising administering to said subject a therapeutically effective amount of a compound selected from the group consisting of cynarine, eravacycline and prexasertib or a pharmaceutically acceptable salt thereof, preferably eravacycline or a pharmaceutically acceptable salt thereof.

In a fourth aspect, the present invention relates to a combination comprising one or more compounds selected from the group consisting of cynarine, eravacycline, prexasertib, remdesivir, ribavirin, chloroquine, hydroxychloroquine, oseltamivir, favipiravir, lopinavir, ritonavir, umifenovir, darunavir, cobicistat, galidesivir, danoprevir, ASC09, baloxavir marboxil, azvudine, vicromax, cidofovir, pleconaril, valacyclovir, abacavir, dolutegravir, lamivudine, emtricitabine, tenofovir, ledipasvir, sofosbuvir, velpatasvir, pimodivir, galidesivir, acyclovir, brivudine, famciclovir, penciclovir, ganciclovir, amantadine, rimantadine, nelfinavir, grazoprevir, atazanavir, voxilaprevir, saquinavir, amprenavir, indinavir, asunaprevir, fosamprenavir, danoprevir, simeprevir, vaniprevir, narlaprevir, boceprevir, tipranavir, glecaprevir, zanamivir, Teicoplanin, oritavancin, dalbavancin, monensin, mycophenolic acid, gemcitabine, Navitoclax, luteolin, glycyrrhizin, eflornithine, silvestrol, emodin, amiodarone, homoharringtonine, alisporivir, hexachlorophene, camostat, memantine, indomethacin, telavancin, trametinib, N3, ebselen, disulfiram, carmofur, shikonin, EIDD-1931, EIDD-2801, ivermectin, tideglusib, PX-12, Hydrocortisone, methylprednisolone, dexamethasone, ciclesonide, naproxen, peginterferon alpha2b, colchicine, opaganib, Novaferon, SNG001, peginterferon lambda, Rebif, sargramostim, anakinra, baricitinib, antibodies from recovered COVID19 patients, bevacizumab, PD-1 antibody, leronlimab, camrelizumab, sarilumab, tocilizumab, gimsilumab, TJM2, lenzilumab, siltuximab, eculizumab, mavrilimumab, canakinumab, namilumab, emapalumab, meplazumab, ruxolitinib, fingolimod, camostat mesylate, ifenprodil, APN01, Brilacidin, BXT-25, nafamostat, losartan, alteplase, AT001, plitidepsin, dipyridamole, ebastine, azithromycin, solnatide, selinexor or a pharmaceutically acceptable salt thereof, for use in the treatment and/or prevention of viral infections by betacoronaviruses, especially by respiratory syndrome-related coronaviruses selected from the species “Middle East respiratory syndrome-related coronavirus” (such as MERS-CoV) and “Severe respiratory syndrome-related coronavirus” (such SARS-CoV and SARS-CoV-2), wherein at least one compound is selected from the group consisting of cynarine, eravacycline and prexasertib or a pharmaceutically acceptable salt thereof, preferably wherein at least one compound is selected from the group consisting of eravacycline and prexasertib or a pharmaceutically acceptable salt thereof.

In a fifth aspect, the invention relates to the use of a combination comprising one or more compounds selected from the group consisting of cynarine, eravacycline, prexasertib, remdesivir, ribavirin, chloroquine, hydroxychloroquine, oseltamivir, favipiravir, lopinavir, ritonavir, umifenovir, darunavir, cobicistat, galidesivir, danoprevir, ASC09, baloxavir marboxil, azvudine, vicromax, cidofovir, pleconaril, valacyclovir, abacavir, dolutegravir, lamivudine, emtricitabine, tenofovir, ledipasvir, sofosbuvir, velpatasvir, pimodivir, galidesivir, acyclovir, brivudine, famciclovir, penciclovir, ganciclovir, amantadine, rimantadine, nelfinavir, grazoprevir, atazanavir, voxilaprevir, saquinavir, amprenavir, indinavir, asunaprevir, fosamprenavir, danoprevir, simeprevir, vaniprevir, narlaprevir, boceprevir, tipranavir, glecaprevir, zanamivir, Teicoplanin, oritavancin, dalbavancin, monensin, mycophenolic acid, gemcitabine, Navitoclax, luteolin, glycyrrhizin, eflornithine, silvestrol, emodin, amiodarone, homoharringtonine, alisporivir, hexachlorophene, camostat, memantine, indomethacin, telavancin, trametinib, N3, ebselen, disulfiram, carmofur, shikonin, EIDD-1931, EIDD-2801, ivermectin, tideglusib, PX-12, Hydrocortisone, methylprednisolone, dexamethasone, ciclesonide, naproxen, peginterferon alpha2b, colchicine, opaganib, Novaferon, SNG001, peginterferon lambda, Rebif, sargramostim, anakinra, baricitinib, antibodies from recovered COVID19 patients, bevacizumab, PD-1 antibody, leronlimab, camrelizumab, sarilumab, tocilizumab, gimsilumab, TJM2, lenzilumab, siltuximab, eculizumab, mavrilimumab, canakinumab, namilumab, emapalumab, meplazumab, ruxolitinib, fingolimod, camostat mesylate, ifenprodil, APN01, Brilacidin, BXT-25, nafamostat, losartan, alteplase, AT001, plitidepsin, dipyridamole, ebastine, azithromycin, solnatide, selinexoror a pharmaceutically acceptable salt thereof, in the manufacture of a medicament for the treatment and/or prevention of viral infections by betacoronaviruses, especially by respiratory syndrome-related coronaviruses selected from the species “Middle East respiratory syndrome-related coronavirus” (such as MERS-CoV) and “Severe respiratory syndrome-related coronavirus” (such as SARS-CoV and SARS-CoV-2), wherein at least one compound is selected from the group consisting of cynarine, eravacycline and prexasertib, or a pharmaceutically acceptable salt thereof, preferably wherein at least one compound is selected from the group consisting of eravacycline and prexasertib or a pharmaceutically acceptable salt thereof.

In a sixth aspect, the invention relates to a method of treating and/or preventing viral infections by betacoronavirus, especially by respiratory syndrome-related coronaviruses selected from the species “Middle East respiratory syndrome-related coronavirus” (such as MERS-CoV) and “Severe respiratory syndrome-related coronavirus” (such as SARS-CoV and SARS-CoV-2) in a subject, comprising administering to said subject a therapeutically effective amount of a combination comprising one or more compounds selected from the group consisting of cynarine, eravacycline, prexasertib, remdesivir, ribavirin, chloroquine, hydroxychloroquine, oseltamivir, favipiravir, lopinavir, ritonavir, umifenovir, darunavir, cobicistat, galidesivir, danoprevir, ASC09, baloxavir marboxil, azvudine, vicromax, cidofovir, pleconaril, valacyclovir, abacavir, dolutegravir, lamivudine, emtricitabine, tenofovir, ledipasvir, sofosbuvir, velpatasvir, pimodivir, galidesivir, acyclovir, brivudine, famciclovir, penciclovir, ganciclovir, amantadine, rimantadine, nelfinavir, grazoprevir, atazanavir, voxilaprevir, saquinavir, amprenavir, indinavir, asunaprevir, fosamprenavir, danoprevir, simeprevir, vaniprevir, narlaprevir, boceprevir, tipranavir, glecaprevir, zanamivir, Teicoplanin, oritavancin, dalbavancin, monensin, mycophenolic acid, gemcitabine, Navitoclax, luteolin, glycyrrhizin, eflornithine, silvestrol, emodin, amiodarone, homoharringtonine, alisporivir, hexachlorophene, camostat, memantine, indomethacin, telavancin, trametinib, N3, ebselen, disulfiram, carmofur, shikonin, EIDD-1931, EIDD-2801, ivermectin, tideglusib, PX-12, Hydrocortisone, methylprednisolone, dexamethasone, ciclesonide, naproxen, peginterferon alpha2b, colchicine, opaganib, Novaferon, SNG001, peginterferon lambda, Rebif, sargramostim, anakinra, baricitinib, antibodies from recovered COVID19 patients, bevacizumab, PD-1 antibody, leronlimab, camrelizumab, sarilumab, tocilizumab, gimsilumab, TJM2, lenzilumab, siltuximab, eculizumab, mavrilimumab, canakinumab, namilumab, emapalumab, meplazumab, ruxolitinib, fingolimod, camostat mesylate, ifenprodil, APN01, Brilacidin, BXT-25, nafamostat, losartan, alteplase, AT001, plitidepsin, dipyridamole, ebastine, azithromycin, solnatide, selinexor or a pharmaceutically acceptable salt thereof, wherein at least one compound is selected from the group consisting of cynarine, eravacycline and prexasertib or a pharmaceutically acceptable salt thereof, preferably wherein at least one compound is selected from the group consisting of eravacycline and prexasertib or a pharmaceutically acceptable salt thereof.

### DESCRIPTION OF THE INVENTION

In the first aspect, the present invention relates to a compound selected from the group consisting of cynarine, eravacycline and prexasertib or a pharmaceutically acceptable salt thereof, preferably eravacycline or a pharmaceutically acceptable salt thereof, for use in the treatment and/or prevention of viral infections by betacoronaviruses, especially by respiratory syndrome-related coronaviruses selected from the species “Middle East respiratory syndrome-related coronavirus” (such as MERS-CoV) and “Severe respiratory syndrome-related coronavirus” (such as SARS-CoV and SARS-CoV-2).

The invention also relates to the use of a compound selected from the group consisting of cynarine, eravacycline and prexasertib or a pharmaceutically acceptable salt thereof, preferably eravacycline or a pharmaceutically acceptable salt thereof, in the manufacture of a medicament for the treatment and/or prevention of viral infections by betacoronaviruses, especially by respiratory syndrome-related coronaviruses selected from the species “Middle East respiratory syndrome-related coronavirus” (such as MERS-CoV) and “Severe respiratory syndrome-related coronavirus” (such SARS-CoV and SARS-CoV-2).

The invention also relates to a method of treating and/or preventing viral infections by betacoronaviruses, especially by respiratory syndrome-related coronaviruses selected from the species “Middle East respiratory syndrome-related coronavirus” (such as MERS-CoV) and “Severe respiratory syndrome-related coronavirus” (such as SARS-CoV and SARS-CoV-2) in a subject, comprising administering to said subject a therapeutically effective amount of a compound selected from the group consisting of cynarine, eravacycline and prexasertib or a pharmaceutically acceptable salt thereof, preferably eravacycline or a pharmaceutically acceptable salt thereof.

The term “betacoronavirus”, as used herein, is used to designate any viral species of the taxonomic genus “Betacoronavirus”, within the realm “Riboviria”, the order: “Nidovirales”, the family “Coronaviridae” and the subfamily “Orthocoronavirinae”.

In a preferred embodiment of the present invention, the betacoronaviruses are selected from the subfamilies “Sarbecovirus” and “Merbecovirus”.

In another embodiment of the present invention the betacoronaviruses are expressing a 3C-like proteinase having an homology of at least 80%, more preferably of at least 85%, still more preferably of at least 90%, still more preferably of at least 95% and still more preferably of at least 98% with the proteinase having the NCBI Reference sequence YP_009725301.1.

In still another embodiment of the present invention the betacoronaviruses express a proteinase able to cleave the substrate KTSAVLQSGFRKME [SEQ ID NO: 1].

The terms “treating” and “treatment”, as used herein, means reversing, alleviating, inhibiting the progress of, the disease or condition to which such term applies, or one or more symptoms of such disease or condition, such as lowering the viral load in a patient with respect to pretreatment levels.

The terms “preventing” and “prevention”, as used herein, means avoiding or inhibiting the onset of one or more symptoms of coronavirus infections such as fever, cough, shortness of breath, muscle pain, sputum production, diarrhea, sore throat, loss of smell, pneumonia and abdominal pain.

Preferably, the compounds disclosed herein, are used for the treatment of viral infections by “Severe respiratory syndrome-related coronavirus” and most preferably for the treatment of viral infections by SARS-CoV-2.

Cynarine or 1,3-bis[[(E)-3-(3,4-dihydroxyphenyl)prop-2-enoyl]oxy]-4,5-dihydroxycyclohexane-1-carboxylic acid has the chemical structure depicted below.

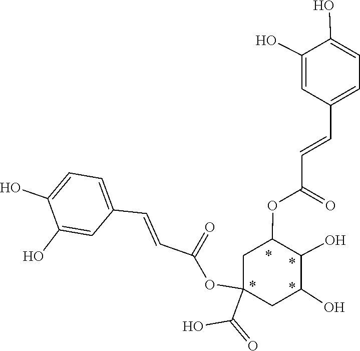 [SMILES: O=C(/C=C/c1ccc(O)c(O)c1)OC1CC(OC(=O)/C=C/c2ccc(O)c(O)c2)(C(=O)O)CC(O)C1O]

Cynarine has 4 chiral centers (marked with an asterisk in the formula above) and, thus, can exist in the form of 16 different stereoisomers, which are all encompassed by the term cynarine in the present invention, in particular the (1R,3R,4S,5R) estereoisomer is encompassed by said term.

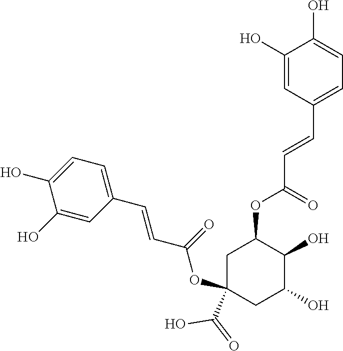 [SMILES: O=C(/C=C/c1ccc(O)c(O)c1)O[C@@H]1C[C@@](OC(=O)/C=C/c2ccc(O)c(O)c2)(C(=O)O)C[C@@H](O)[C@@H]1O]

This compound has been developed as an agent which promotes the discharge of bile from the system This compound is commercially available or may be synthesized using a suitable preparation method, such as that disclosed in U.S. Pat. No. 3,100,224 A.

Eravacycline or (4S,4aS,5aR,12aS)-4-(Dimethylamido)-7-fluoro-3,10,12,12a-tetrahydroxy-1,11-dioxo-9-[(1-pyrrolidinylacetyl)amino]-1,4,4a,5,5a,6,11,12a-octahydro-2-tetracenecarboxamide has the chemical structure depicted below.

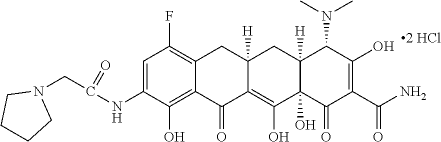 [SMILES: CN(C)[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

This compound was developed as a broad spectrum antibiotic, in particular for the treatment of complicated intra-abdominal infections. This compound is commercially available or may be synthesized using a suitable preparation method, such as that disclosed in Xiao et. al. “Fluorocyclines. 1. 7-Fluoro-9-pyrrolidinoacetamido-6-demethyl-6-deoxytetracycline: A Potent, Broad Spectrum Antibacterial Agent” J. Med. Chem. 2012, 55, 597-605.

Prexasertib or 5-((5-(2-(3-Aminopropoxy)-6-methoxyphenyl)-1H-pyrazol-3-yl)amino)-2-pyrazinecarbonitrile has the chemical structure depicted below.

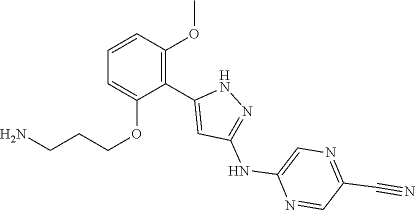 [SMILES: COc1cccc(OCCCN)c1-c1cc(Nc2cnc(C#N)cn2)n[nH]1]

This compound is in development for the treatment of recurrent medulloblastoma. This compound is commercially available or may be synthesized using a suitable preparation method, such as that disclosed in Cole et al. “Kilogram-scale prexasertib monolactate monohydrate synthesis under continuous-flow CGMP conditions” Science 2017, 356, 6343, 1144-1150.

The term “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

Further, the term “pharmaceutically acceptable salt” refers to any salt, which, upon administration to the recipient is capable of providing (directly or indirectly) a compound as described herein. For instance, a pharmaceutically acceptable salt of compounds provided herein may be acid addition salts, base addition salts or metallic salts, and they can be synthesized from the parent compound, which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, ammonium, and organic alkali salts such as, for example, ethylenediamine, ethanolamine, N,N-dialkylenethanolamine, triethanolamine, glucamine and basic aminoacids salts. Examples of the metallic salts include, for example, sodium, potassium, calcium, magnesium, aluminium and lithium salts.

In another particular embodiment, the compound for use according to the invention is cynarine or a pharmaceutically acceptable salt thereof, preferably cynarine (i.e. cynarine free acid).

In a particular embodiment, the compound for use according to the invention is eravacycline or a pharmaceutically acceptable salt thereof, preferably eravacycline dihydrochloride.

In another particular embodiment, the compound for use according to the invention is prexasertib or a pharmaceutically acceptable salt thereof, preferably prexasertib dihydrochloride or prexasertib monomesylate monohydrate, more preferably prexasertib monomesylate monohydrate.

In another particular embodiment, the viral infection is an infection by “Severe respiratory syndrome-related coronavirus” and most preferably by SARS-CoV-2.

The compounds for use according to the invention may be administered by any appropriate route (via), such as, oral (e.g., oral, sublingual, etc.), parenteral (e.g., subcutaneous, intramuscular, intravenous, etc.), vaginal, rectal, nasal, topical, ophtalmic, inhaled, intranasal, intratracheal, pulmonary, etc., preferably oral, inhaled or parenteral, more preferably intravenous.

The compounds for use according to the invention may, in particular be administered by inhalation. In this case, they are advantageously formulated as dry powder compositions and may, for example, be presented in capsules and cartridges of for example gelatine or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred.

In particular, the compounds for use according to the invention are administered as a pharmaceutical composition, which comprises the corresponding (active) compound and one or more pharmaceutically acceptable excipients.

The term “pharmaceutically acceptable excipient” refers to a vehicle, diluent, or adjuvant that is administered with the active ingredient. Such pharmaceutical excipients can be sterile liquids, such as water and oils, including those of petroleum, animal, vegetable, or synthetic origin, such as peanut oil, soybean oil, mineral oil, sesame oil, and similars. Water or saline aqueous solutions and aqueous dextrose and glycerol solutions, particularly for injectable solutions, are preferably used as vehicles. Suitable pharmaceutical vehicles are known by the skilled person.

The pharmaceutically acceptable excipient necessary to manufacture the desired pharmaceutical composition of the invention will depend, among other factors, on the elected administration route. Said pharmaceutical compositions may be manufactured according to conventional methods known by the skilled person in the art.

The compounds for use according to the invention may be administered in a “therapeutically effective amount”, i.e. a nontoxic but sufficient amount of the corresponding compound to provide the desired effect. The amount that is “effective” will vary from subject to subject, depending on the age and general condition of the individual, the particular compound administered, and the like. Thus, it is not always possible to specify an exact “therapeutically effective amount”. However, an appropriate amount in any individual case may be determined by one of ordinary skill in the art using routine experimentation.

The compounds for use according to the invention will typically be administered once or more times a day, for example 1, 2, 3 or 4 times daily, with typical total daily doses depending on the particular compound and severity of the disease, and may be easily determined by the skilled practitioner.

By way of example, typical total daily doses of cynarine or a pharmaceutically acceptable salt thereof are in the range of from 0.1 to 2000 mg/day (expressed as cynarine free base), preferably from 1 to 600 mg/ day, even more preferably from 1 to 100 mg/day.

Typical total daily doses of eravacycline or a pharmaceutically acceptable salt thereof administered by oral route are in the range of from 0.1 to 3000 mg/day (expressed as eravacycline free base), preferably from 0.1 to 1000 mg/day, even more preferably from 1 to 500 mg/day. Typical total daily doses of eravacycline or a pharmaceutically acceptable salt thereof administered by parenteral or inhaled route are in the range of from 0.1 to 20 mg/kg/day (expressed as eravacycline free base), preferably from 0.1 to 5 mg/kg/day, even more preferably from 0.1 to 2 mg/kg/day.

Typical total daily doses of prexasertib or a pharmaceutically acceptable salt thereof are in the range of from 0.1 to 2000 mg/day (expressed as prexasertib free base), preferably from 0.1 to 200 mg/ day, even more preferably from 1 to 20 mg/day.

The pharmaceutical compositions may be prepared using standard methods such as those described or referred to in the Spanish and US Pharmacopoeias and similar reference texts.

The term “subject” refers to a mammal, e.g., a human. The compounds for use according to the invention may be administered as the sole active ingredient or in combination with other active ingredients. In a particular embodiment, the compounds are used as the sole active ingredient. In another particular embodiment, the compounds are used in combination with other active ingredients.

In another aspect, the present invention relates to a combination comprising one or more compounds selected from the group consisting of cynarine, eravacycline, prexasertib, remdesivir, ribavirin, chloroquine, hydroxychloroquine, oseltamivir, favipiravir, lopinavir, ritonavir, umifenovir, darunavir, cobicistat, galidesivir, danoprevir, ASC09, baloxavir marboxil, azvudine, vicromax, cidofovir, pleconaril, valacyclovir, abacavir, dolutegravir, lamivudine, emtricitabine, tenofovir, ledipasvir, sofosbuvir, velpatasvir, pimodivir, galidesivir, acyclovir, brivudine, famciclovir, penciclovir, ganciclovir, amantadine, rimantadine, nelfinavir, grazoprevir, atazanavir, voxilaprevir, saquinavir, amprenavir, indinavir, asunaprevir, fosamprenavir, danoprevir, simeprevir, vaniprevir, narlaprevir, boceprevir, tipranavir, glecaprevir, zanamivir, teicoplanin, oritavancin, dalbavancin, monensin, mycophenolic acid, gemcitabine, navitoclax, luteolin, glycyrrhizin, eflornithine, silvestrol, emodin, amiodarone, homoharringtonine, alisporivir, hexachlorophene, camostat, memantine, indomethacin, telavancin, trametinib, N3, ebselen, disulfiram, carmofur, shikonin, EIDD-1931, EIDD-2801, ivermectin, tideglusib, PX-12, hydrocortisone, methylprednisolone, dexamethasone, ciclesonide, naproxen, peginterferon alpha2b, colchicine, opaganib, Novaferon, SNG001, peginterferon lambda, Rebif, sargramostim, anakinra, baricitinib, antibodies from recovered COVID19 patients, bevacizumab, PD-1 antibody, leronlimab, camrelizumab, sarilumab, tocilizumab, gimsilumab, TJM2, lenzilumab, siltuximab, eculizumab, mavrilimumab, canakinumab, namilumab, emapalumab, meplazumab, ruxolitinib, fingolimod, camostat mesylate, ifenprodil, APN01, brilacidin, BXT-25, nafamostat, losartan, alteplase, AT001, plitidepsin, dipyridamole, ebastine, azithromycin, solnatide, selinexor or a pharmaceutically acceptable salt thereof, for use in the treatment and/or prevention of viral infections by coronavirus, especially by respiratory syndrome-related coronavirus selected from the species “Middle East respiratory syndrome-related coronavirus” (such as MERS-CoV) and “Severe respiratory syndrome-related coronavirus” (such as SARS-CoV and SARS-CoV-2), wherein at least one compound is selected from the group consisting of cynarine, eravacycline and prexasertib or a pharmaceutically acceptable salt thereof, preferably wherein at least one compound is selected from the group consisting of eravacycline and prexasertib or a pharmaceutically acceptable salt thereof.

The invention also relates to the use of a combination comprising one or more compounds selected from the group consisting of cynarine, eravacycline, prexasertib, remdesivir, ribavirin, chloroquine, hydroxychloroquine, oseltamivir, favipiravir, lopinavir, ritonavir, umifenovir, darunavir, cobicistat, galidesivir, danoprevir, ASC09, baloxavir marboxil, azvudine, vicromax, cidofovir, pleconaril, valacyclovir, abacavir, dolutegravir, lamivudine, emtricitabine, tenofovir, ledipasvir, sofosbuvir, velpatasvir, pimodivir, galidesivir, acyclovir, brivudine, famciclovir, penciclovir, ganciclovir, amantadine, rimantadine, nelfinavir, grazoprevir, atazanavir, voxilaprevir, saquinavir, amprenavir, indinavir, asunaprevir, fosamprenavir, danoprevir, simeprevir, vaniprevir, narlaprevir, boceprevir, tipranavir, glecaprevir, zanamivir, teicoplanin, oritavancin, dalbavancin, monensin, mycophenolic acid, gemcitabine, navitoclax, luteolin, glycyrrhizin, eflornithine, silvestrol, emodin, amiodarone, homoharringtonine, alisporivir, hexachlorophene, camostat, memantine, indomethacin, telavancin, trametinib, N3, ebselen, disulfiram, carmofur, shikonin, EIDD-1931, EIDD-2801, ivermectin, tideglusib, PX-12, hydrocortisone, methylprednisolone, dexamethasone, ciclesonide, naproxen, peginterferon alpha2b, colchicine, opaganib, Novaferon, SNG001, peginterferon lambda, Rebif, sargramostim, anakinra, baricitinib, antibodies from recovered COVID19 patients, bevacizumab, PD-1 antibody, leronlimab, camrelizumab, sarilumab, tocilizumab, gimsilumab, TJM2, lenzilumab, siltuximab, eculizumab, mavrilimumab, canakinumab, namilumab, emapalumab, meplazumab, ruxolitinib, fingolimod, camostat mesylate, ifenprodil, APN01, brilacidin, BXT-25, nafamostat, losartan, alteplase, AT001, plitidepsin, dipyridamole, ebastine, azithromycin, solnatide, selinexor or a pharmaceutically acceptable salt thereof, in the manufacture of a medicament for the treatment and/or prevention of viral infections by coronavirus, especially by respiratory syndrome-related coronavirus selected from the species “Middle East respiratory syndrome-related coronavirus” (such as MERS-CoV) and “Severe respiratory syndrome-related coronavirus” (such as SARS-CoV and SARS-CoV-2), wherein at least one compound is selected from the group consisting of cynarine, eravacycline and prexasertib or a pharmaceutically acceptable salt thereof, preferably wherein at least one compound is selected from the group consisting of eravacycline and prexasertib or a pharmaceutically acceptable salt thereof.

The invention also relates a method of treating and/or preventing viral infections by coronavirus, especially by respiratory syndrome-related coronavirus selected from the species “Middle East respiratory syndrome-related coronavirus” (such as MERS-CoV) and “Severe respiratory syndrome-related coronavirus” (such as SARS-CoV and SARS-CoV-2) in a subject, comprising administering to said subject a therapeutically effective amount of a combination comprising one or more compounds selected from the group consisting of cynarine, eravacycline, prexasertib, remdesivir, ribavirin, chloroquine, hydroxychloroquine, oseltamivir, favipiravir, lopinavir, ritonavir, umifenovir, darunavir, cobicistat, galidesivir, danoprevir, ASC09, baloxavir marboxil, azvudine, vicromax, cidofovir, pleconaril, valacyclovir, abacavir, dolutegravir, lamivudine, emtricitabine, tenofovir, ledipasvir, sofosbuvir, velpatasvir, pimodivir, galidesivir, acyclovir, brivudine, famciclovir, penciclovir, ganciclovir, amantadine, rimantadine, nelfinavir, grazoprevir, atazanavir, voxilaprevir, saquinavir, amprenavir, indinavir, asunaprevir, fosamprenavir, danoprevir, simeprevir, vaniprevir, narlaprevir, boceprevir, tipranavir, glecaprevir, zanamivir, teicoplanin, oritavancin, dalbavancin, monensin, mycophenolic acid, gemcitabine, navitoclax, luteolin, glycyrrhizin, eflornithine, silvestrol, emodin, amiodarone, homoharringtonine, alisporivir, hexachlorophene, camostat, memantine, indomethacin, telavancin, trametinib, N3, ebselen, disulfiram, carmofur, shikonin, EIDD-1931, EIDD-2801, ivermectin, tideglusib, PX-12, hydrocortisone, methylprednisolone, dexamethasone, ciclesonide, naproxen, peginterferon alpha2b, colchicine, opaganib, Novaferon, SNG001, peginterferon lambda, Rebif, sargramostim, anakinra, baricitinib, antibodies from recovered COVID19 patients, bevacizumab, PD-1 antibody, leronlimab, camrelizumab, sarilumab, tocilizumab, gimsilumab, TJM2, lenzilumab, siltuximab, eculizumab, mavrilimumab, canakinumab, namilumab, emapalumab, meplazumab, ruxolitinib, fingolimod, camostat mesylate, ifenprodil, APN01, brilacidin, BXT-25, nafamostat, losartan, alteplase, AT001, plitidepsin, dipyridamole, ebastine, azithromycin, solnatide, selinexor or a pharmaceutically acceptable salt thereof wherein at least one compound is selected from the group consisting of cynarine, eravacycline and prexasertib or a pharmaceutically acceptable salt thereof, preferably wherein at least one compound is selected from the group consisting of eravacycline and prexasertib or a pharmaceutically acceptable salt thereof.

The term “combination” refers to a product comprising one or more of the defined compounds, either in a single composition or in several compositions (or units), in which case the corresponding compounds are distributed among the several compositions. Preferably, the combination refers to several compositions, in particular comprising one composition (or unit) per compound (compound as defined above) of the combination. The expression “one or more” when characterizing the combination refers to at least one, preferably 1, 2, 3, 4, or 5 compounds, more preferably, 1, 2 or 3 compounds, even more preferably 1 or 2 compounds.

When the combination is in the form of a single composition, the compounds present in the combination are always administered simultaneously.

When the combination is in the form of several compositions (or units), each of them having at least one of the compounds of the combination, the compositions or (units) may be administered simultaneously, sequentially or separately.

Simultaneous administration means that the compounds or compositions (or units) are administered at the same time.

Sequential administration means that the compounds or compositions (or units) are administered at different time points in a chronologically staggered manner.

Separate administration means that the compounds or compositions (or units) are administered at different time points independently of each other.

Cynarine, eravacycline and prexasertib, including their pharmaceutically acceptable salts have been described in detail above.

Remdesivir or (2S)-2-{(2R,3S,4R,5R)-[5-(4-Aminopyrrolo[2,1-f][1,2,4]triazin-7-yl)-5-cyano-3,4-dihydroxy-tetrahydro-furan-2-ylmethoxy]phenoxy-(S)-phosphorylamino}propionic acid 2-ethyl-butyl ester has the chemical structure depicted below.

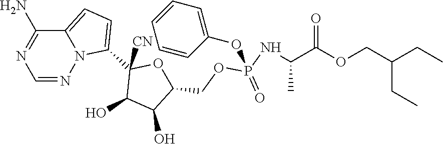

This compound is being developed as a broad spectrum antiviral. This compound is commercially available or may be synthesized using a suitable preparation method, such as that disclosed in WO 2017/184668 A1.

Ribavirin or 1-[(2R,3R,4S,5R)-3,4-dihydroxy-5-(hydroxymethyl)oxolan-2-yl]-1,2,4-triazole-3-carboxamide has the chemical structure depicted below.

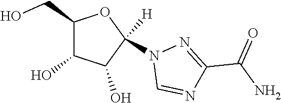 [SMILES: NC(=O)c1ncn([C@@H]2O[C@H](CO)[C@@H](O)[C@H]2O)n1]

This compound is used as a broad spectrum antiviral. This compound is commercially available or may be synthesized using a suitable preparation method, such as that disclosed in DE 2 220 246.

Chloroquine or (RS)-N′-(7-chloroquinolin-4-yl)-N,N-diethyl-pentane-1,4-diamine has the chemical structure depicted below.

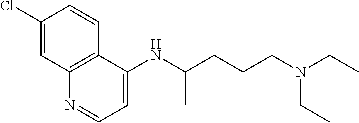 [SMILES: CCN(CC)CCCC(C)Nc1ccnc2cc(Cl)ccc12]

This compound is used as an antimalarial agent and for the treatment of autoimmune diseases such as lupus. This compound is commercially available or may be synthesized using a suitable preparation method, such as that disclosed in DE 683 692.

Hydroxychloroquine or (RS)-2-[{4-[(7-chloroquinolin-4-yl)amino]pentyl}(ethyl)amino]ethanol has the chemical structure depicted below.

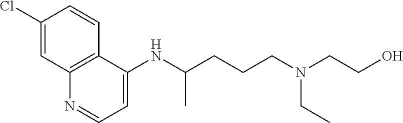 [SMILES: CCN(CCO)CCCC(C)Nc1ccnc2cc(Cl)ccc12]

This compound is used as an antimalarial agent and for the treatment of autoimmune diseases such as lupus. This compound is commercially available or may be synthesized using a suitable preparation method, such as that disclosed in U.S. Pat. No. 2,546,658.

Oseltamivir or ethyl (3R,4R,5S)-4-acetamido-5-amino-3-pentan-3-yloxycyclohex-1-ene-1-carboxylate has the chemical structure depicted below.

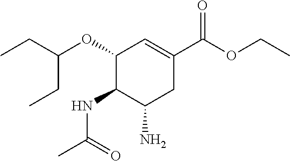 [SMILES: CCOC(=O)C1=C[C@@H](OC(CC)CC)[C@H](NC(C)=O)[C@@H](N)C1]

This compound is used for the treatment of influenza virus infections with the brand name Tamiflu. This compound is commercially available or may be synthesized using a suitable preparation method, such as that disclosed in U.S. Pat. No. 5,763,483.

Favipiravir or 6-Fluoro-3-hydroxypyrazine-2-carboxamide has the chemical structure depicted below.

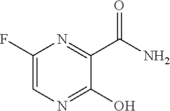 [SMILES: NC(=O)c1nc(F)cnc1O]

This compound, also known as Favilavir or T705, is used for the treatment of influenza virus infections with the brand name Avigan. This compound is commercially available or may be synthesized using a suitable preparation method, such as that disclosed in WO 00/10569 A1.

Lopinavir or (2S)-N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide has the chemical structure depicted below.

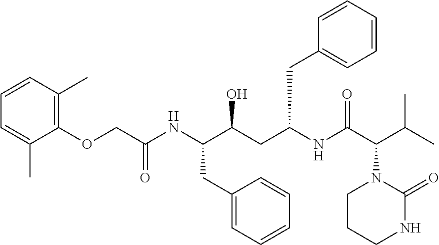 [SMILES: Cc1cccc(C)c1OCC(=O)N[C@@H](Cc1ccccc1)[C@@H](O)C[C@H](Cc1ccccc1)NC(=O)[C@H](C(C)C)N1CCCNC1=O]

This compound is an inhibitor of the HIV protease and is used for the treatment of HIV infections. This compound is commercially available or may be synthesized using a suitable preparation method, such as that disclosed in WO 97/21685 A1.

Ritonavir or 1,3-thiazol-5-ylmethyl N-[(2S,3S,5S)-3-hydroxy-5-[[(2S)-3-methyl-2-{[[methyl-[(2-propan-2-yl-1,3-thiazol-4-yl)methyl]carbamoyl]amino]butanoyl]amino]-1,6-diphenylhexan-2-yl]carbamate has the chemical structure depicted below.

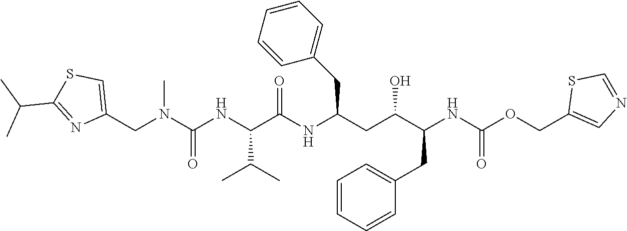 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

This compound is an inhibitor of CYP3A4 and it is used in combination with lopinavir or with other antivirals for the treatment of HIV infections. This compound is commercially available or may be synthesized using a suitable preparation method, such as that disclosed in EP 0 402 646 A1.

Umifenovir or Ethyl 6-bromo-4-[(dimethylamino)methyl]-5-hydroxy-1-methyl-2-[(phenylthio)methyl]-1H-indole-3-carboxylate has the chemical structure depicted below.

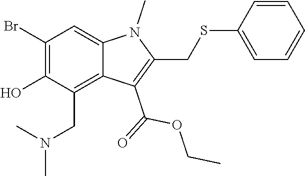 [SMILES: CCOC(=O)c1c(CSc2ccccc2)n(C)c2cc(Br)c(O)c(CN(C)C)c12]

This compound is an antiviral used for the treatment of influenza virus infections. This compound is commercially available or may be synthesized using a suitable preparation method, such as that disclosed in WO 90/08135 A1.

Darunavir or [(1R,5S,6R)-2,8-dioxabicyclo[3.3.0]oct-6-yl] N-[(2S,3R)-4-[(4-aminophenyl)sulfonyl-(2-methylpropyl)amino]-3-hydroxy-1-phenyl-butan-2-yl] carbamate has the chemical structure depicted below.

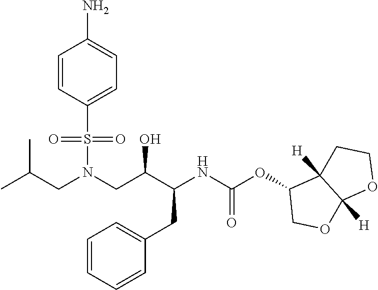 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

This compound is an HIV protease inhibitor used for the treatment of HIV infections. This compound is commercially available or may be synthesized using a suitable preparation method, such as that disclosed in WO 2004/033462 A2.

Cobicistat or 1,3-thiazol-5-ylmethyl N-[(2R,5R)-5-[[(2S)-2-[[methyl-[(2-propan-2-yl-1,3-thiazol-4-yl)methyl]carbamoyl]amino]-4-morpholin-4-ylbutanoyl]amino]-1,6-diphenylhexan-2-yl]carbamate has the chemical structure depicted below.

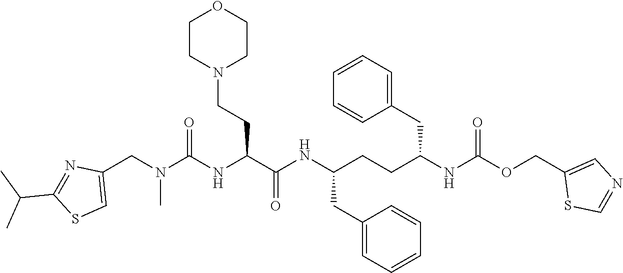 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

This compound is an inhibitor of CYP3A that is used in combination with darunavir or with other antivirals for the treatment of HIV infections. This compound is commercially available or may be synthesized using a suitable preparation method, such as that disclosed in WO 2008/103949 A1.

Galidesivir or (2S,3S,4R,5R)-2-(4-Amino-5H-pyrrolo[3,2-d]pyrimidin-7-yl)-5-(hydroxymethyl)-3,4-pyrrolidinediol has the chemical structure depicted below.

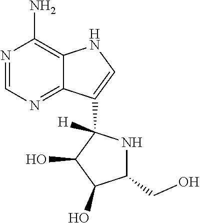 [SMILES: Nc1ncnc2c([C@@H]3N[C@H](CO)[C@@H](O)[C@H]3O)c[nH]c12]

This compound is a nucleoside analog used as a broad spectrum antiviral. This compound is commercially available or may be synthesized using a suitable preparation method, such as that disclosed in U.S. Pat. No. 6,458,799.

Danoprevir or (2R,6S,12Z,13aS,14aR,16aS)-6-[(tert-Butoxycarbonyl)amino]-14a-[N-(cyclopropanesulfonyl)carbamoyl]-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 4-fluoro-1,3-dihydro-2H-isoindole-2-carboxylate 2H— has the chemical structure depicted below.

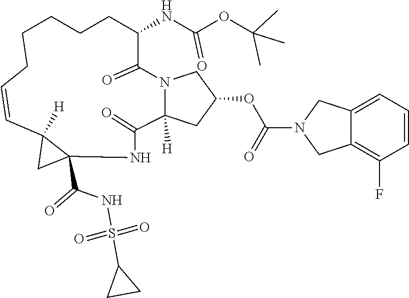

This compound is hepatitis C virus (HCV) NS3/4A protease inhibitor used for the treatment of HCV infections. This compound is commercially available or may be synthesized using a suitable preparation method, such as that disclosed in WO 2005/037214 A2.

ASCO9 (also know as TMC-310911) or [(3aS,4R,6aR)-2,3,3a,4,5,6a-hexahydrofuro[2,3-b]furan-4-yl]-N-[(2S,3R)-4-[[2-[(1-cyclopentylpiperidin-4-yl)amino]-1,3-benzothiazol-6-yl]sulfonyl-(2-methylpropyl)amino]-3-hydroxy-1-phenylbutan-2-yl]carbamate has the chemical structure depicted below

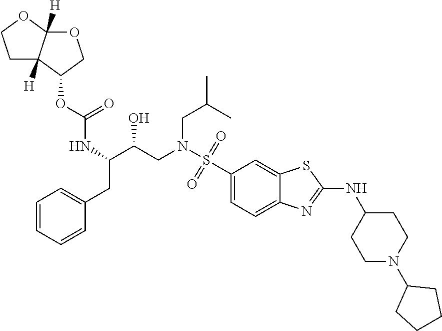 [SMILES: CC(C)CN(C[C@@H](O)[C@H](Cc1ccccc1)NC(=O)O[C@H]1CO[C@H]2OCC[C@H]21)S(=O)(=O)c1ccc2nc(NC3CCN(C4CCCC4)CC3)sc2c1]

This compound is an HIV protease inhibitor that is being developed for the treatment of HIV infections. This compound is commercially available or may be synthesized using a suitable preparation method, such as that disclosed in WO 2007/147884 A1.

Baloxavir marboxil or ({(12aR)-12-[(11S)-7,8-difluoro-6,11-dihydrodibenzo[b,e]thiepin-11-yl]-6,8-dioxo-3,4,6,8,12,12a-hexahydro-1H-[1,4]oxazino[3,4-c]pyrido[2,1-f][1,2,4]triazin-7-yl}oxy)methyl methyl carbonate has the chemical structure depicted below.

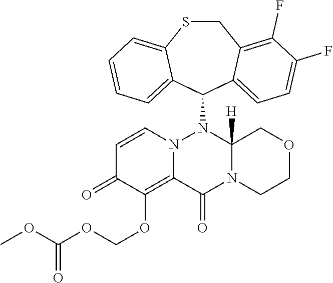 [SMILES: COC(=O)OCOc1c2n(ccc1=O)N([C@@H]1c3ccccc3SCc3c1ccc(F)c3F)[C@@H]1COCCN1C2=O]

This compound is used for the treatment of influenza virus infections. This compound is commercially available or may be synthesized using a suitable preparation method, such as that disclosed in EP 3 290 424 A1.

Azvudine (RO 0622) or 4-amino-1-[(2R,3S,4R,5R)-5-azido-3-fluoro-4-hydroxy-5-(hydroxymethyl)oxolan-2-yl]pyrimidin-2-one has the chemical structure depicted below.

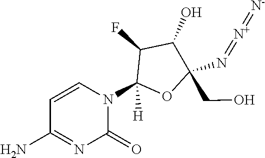 [SMILES: [N-]=[N+]=N[C@]1(CO)O[C@@H](n2ccc(N)nc2=O)[C@@H](F)[C@@H]1O]

This compound is in development for treatment of HIV infections. This compound is commercially available or may be synthesized using a suitable preparation method, such as that disclosed in EP 2 177 527 A1.

Merimepodib (also known as Vicromax) or [(3S)-oxolan-3-yl] N-[[3-[[3-methoxy-4-(1,3-oxazol-5-yl)phenyl]carbamoylamino]phenyl]methyl]carbamate, a broad-spectrum antiviral agent that is being developed by BioSig Technologies for the treatment of COVID19 has the chemical structure depicted below:

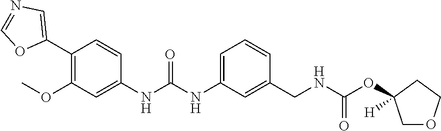 [SMILES: COc1cc(NC(=O)Nc2cccc(CNC(=O)O[C@H]3CCOC3)c2)ccc1-c1cnco1]

This compound is commercially available or may be synthesized using a suitable preparation method, such as that disclosed in WO 97/40028 A1.

Cidofovir or ({[(S)-1-(4-amino-2-oxo-1,2-dihydropyrimidin-1-yl)-3-hydroxypropan-2-yl]oxy}methyl)phosphonic acid has the chemical structure depicted below.

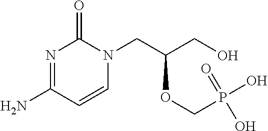 [SMILES: Nc1ccn(C[C@@H](CO)OCP(=O)(O)O)c(=O)n1]

This compound is a base analogue used for the treatment of Citomegalovirus (CMV) infections. This compound is commercially available or may be synthesized using a suitable preparation method, such as that disclosed in EP 0 253 412 A2.

Pleconaril or 3-{3,5-dimethyl-4-[3-(3-methylisoxazol-5-yl)propoxy] phenyl}-5-(trifluoromethyl)-1,2,4-oxadiazole has the chemical structure depicted below.

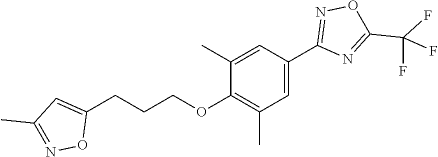 [SMILES: Cc1cc(CCCOc2c(C)cc(-c3noc(C(F)(F)F)n3)cc2C)on1]

This compound, also known as picovir, is used for the treatment of infections by virus of the picornaviridae family. This compound is commercially available or may be synthesized using a suitable preparation method, such as that disclosed in EP 0 566 199 A1.

Valacyclovir or (S)-2-[(2-amino-6-oxo-6,9-dihydro-3H-purin-9-yl)methoxy]ethyl-2-amino-3-methylbutanoate has the chemical structure depicted below.

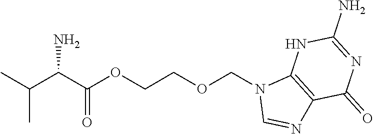 [SMILES: CC(C)[C@H](N)C(=O)OCCOCn1cnc2c(=O)nc(N)[nH]c21]

This compound is a broad spectrum antiviral used for the treatment of herpes virus infections. This compound is commercially available or may be synthesized using a suitable preparation method, such as that disclosed in WO 96/22291 A1.

Abacavir or {(1S,4R)-4-[2-Amino-6-(cyclopropylamino)-9H-purin-9-yl]-2-cyclopent-2-en-1-yl}-methanol has the chemical structure depicted below.

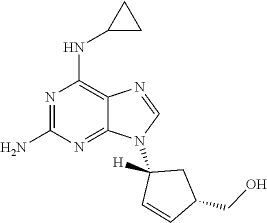 [SMILES: Nc1nc(NC2CC2)c2ncn([C@H]3C=C[C@@H](CO)C3)c2n1]

This compound is a nucleoside analogue used for the treatment of HIV infections. This compound is commercially available or may be synthesized using a suitable preparation method, such as that disclosed in EP 0 434 450 A2.

Dolutegravir or (4R,12a S)-N-(2,4-difluorobenzyl)-7-hydroxy-4-methyl-6,8-dioxo-3,4,6,8,12,12a-hexahydro-2H-pyrido[1′,2′:4,5]pyrazino[2,1-b][1,3]oxazine-9-carboxamide has the chemical structure depicted below.

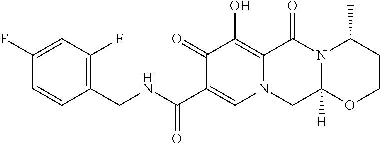 [SMILES: C[C@@H]1CCO[C@H]2Cn3cc(C(=O)NCc4ccc(F)cc4F)c(=O)c(O)c3C(=O)N12]

This compound is an antiviral used for the treatment of HIV infections. This compound is commercially available or may be synthesized using a suitable preparation method, such as that disclosed in WO 2006/116764 A1.

Lamivudine or 4-Amino-1-[(2R,5S)-2-(hydroxymethyl)-1,3-oxathiolan-5-yl]-1,2-dihydropyrimidin-2-one has the chemical structure depicted below.

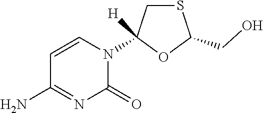 [SMILES: Nc1ccn([C@@H]2CS[C@H](CO)O2)c(=O)n1]

This compound is a nucleoside analogue used for the treatment of HIV infections. This compound is commercially available or may be synthesized using a suitable preparation method, such as that disclosed in WO 91/17159 A1.

Emtricitabine or 4-amino-5-fluoro-1-[(2R,5S)-2-(hydroxymethyl)-1,3-oxathiolan-5-yl]-1,2-dihydropyrimidin-2-one has the chemical structure depicted below.

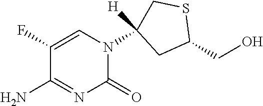

This compound is a nucleoside analogue used for the treatment of HIV infections. This compound is commercially available or may be synthesized using a suitable preparation method, such as that disclosed in WO 92/14743 A2.

Tenofovir or ({[(2R)-1-(6-amino-9H-purin-9-yl)propan-2-yl]oxy}methyl)phosphonic acid has the chemical structure depicted below.

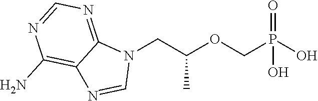 [SMILES: C[C@H](Cn1cnc2c(N)ncnc21)OCP(=O)(O)O]

This compound is a nucleoside analogue used for the treatment of HIV infections. This compound is commercially available or may be synthesized using a suitable preparation method, such as that disclosed in WO 94/03467 A2.

Ledipasvir or methyl N-[(2S)-1-[(6S)-6-[5-[9,9-Difluoro-7-[2-[(1S,2S,4R)-3-[(2S)-2-(methoxycarbonylamino)-3-methylbutanoyl]-3-azabicyclo[2.2.1]heptan-2-yl]-3H-benzimidazol-5-yl]fluoren-2-yl]-1H-imidazol-2-yl]-5-azaspiro[2.4]heptan-5-yl]-3-methyl-1-oxobutan-2-yl]carbamate has the chemical structure depicted below.

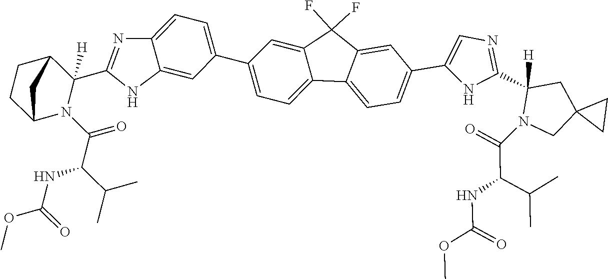 [SMILES: COC(=O)N[C@H](C(=O)N1CC2(CC2)C[C@H]1c1ncc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)[nH]1)C(C)C]

This compound is an antiviral used for the treatment of HCV infections. This compound is commercially available or may be synthesized using a suitable preparation method, such as that disclosed in WO 2010/132601 A1.

Sofosbuvir or Isopropyl (2S)-2-[[[(2R,3R,4R,5R)-5-(2,4-dioxopyrimidin-1-yl)-4-fluoro-3-hydroxy-4-methyl-tetrahydrofuran-2-yl]methoxy-phenoxy-phosphoryl]amino]propanoate has the chemical structure depicted below.

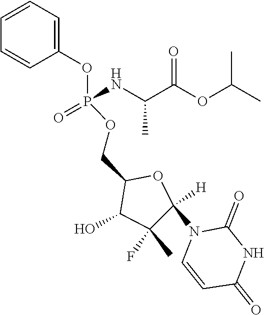 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

This compound is an antiviral used for the treatment of HCV infections. This compound is commercially available or may be synthesized using a suitable preparation method, such as that disclosed in WO 2010/135569 A1.

Velpatasvir or methyl {(2S)-1-[(2S,5S)-2-(9-{2-[(2S,4S)-1-(2R)-2-[(methoxycarbonyl)amino]-2-phenylacetyl}-4-(methoxymethyl)-2-pyrrolidinyl]-1H-imidazol-4-yl}-1,11-dihydroi sochromeno[4′,3′:6,7]naphtho[1,2-d]imidazol-2-yl)-5-methyl-1-pyrrolidinyl]-3-methyl-1-oxo-2-butanylIcarbamate has the chemical structure depicted below.

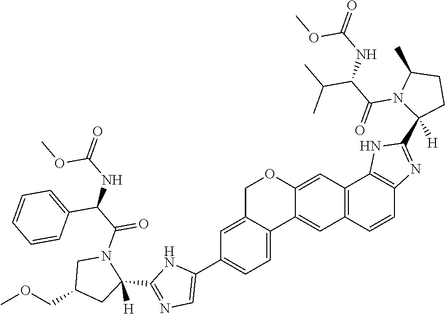 [SMILES: COC[C@H]1C[C@@H](c2ncc(-c3ccc4c(c3)COc3cc5c(ccc6nc([C@@H]7CC[C@H](C)N7C(=O)[C@@H](NC(=O)OC)C(C)C)[nH]c65)cc3-4)[nH]2)N(C(=O)[C@H](NC(=O)OC)c2ccccc2)C1]

This compound is an antiviral used for the treatment of HCV infections. This compound is commercially available or may be synthesized using a suitable preparation method, such as that disclosed in WO 2012/068234 A2.

Pimodivir or (2S,3S)-3-[[5-Fluoro-2-(5-fluoro-1H-pyrrolo[2,3-b]pyridin-3-yl)-4-pyrimidinyl]amino]bicyclo[2.2.2]octane-2-carboxylic acid has the chemical structure depicted below.

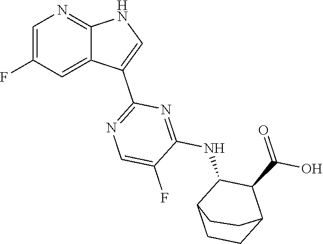 [SMILES: O=C(O)[C@H]1C2CCC(CC2)[C@@H]1Nc1nc(-c2c[nH]c3ncc(F)cc23)ncc1F]

This compound is an antiviral in clinical development for the treatment of influenza virus infections. This compound is commercially available or may be synthesized using a suitable preparation method, such as that disclosed in WO 2015/073481 A1.

Galidesivir or (2S,3S,4R,5R)-2-(4-amino-5H-pyrrolo[3,2-d]pyrimidin-7-yl)-5-(hydroxymethyl) pyrrolidine-3,4-diol has the chemical structure depicted below.

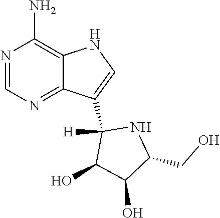 [SMILES: Nc1ncnc2c([C@@H]3N[C@H](CO)[C@@H](O)[C@H]3O)c[nH]c12]

This compound, also known as BCX4430, is a broad spectrum antiviral in clinical development. This compound is commercially available or may be synthesized using a suitable preparation method, such as that disclosed in U.S. Pat. No. 6,458,799 B1.

Acyclovir or 2-Amino-1,9-dihydro-9-((2-hydroxyethoxy)methyl)-3H-purin-6-one has the chemical structure depicted below.

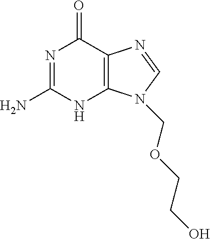 [SMILES: Nc1nc(=O)c2ncn(COCCO)c2[nH]1]

This compound is a broad spectrum antiviral used for the treatment of herpes virus infections. This compound is commercially available or may be synthesized using a suitable preparation method, such as that disclosed in DE 25 39 963 A1.

Brivudine or 5-[(E)-2-bromoethenyl]-1-[(2R,4S,5R)-4-hydroxy-5-(hydroxymethyl)oxolan-2-yl]-1,2,3,4-tetrahydropyrimidine-2,4-dione has the chemical structure depicted below.

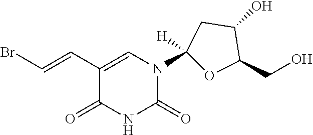 [SMILES: O=c1[nH]c(=O)n([C@H]2C[C@H](O)[C@@H](CO)O2)cc1/C=C/Br]

This compound is a broad spectrum antiviral used for the treatment of herpes virus infections. This compound is commercially available or may be synthesized using a suitable preparation method, such as that disclosed in DE 29 15 254 A1.

Famciclovir or 2-[(acetyloxy)methyl]-4-(2-amino-9H-purin-9-yl)butyl acetate has the chemical structure depicted below.

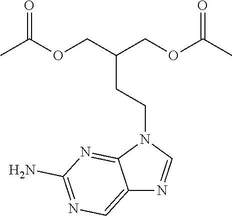 [SMILES: CC(=O)OCC(CCn1cnc2cnc(N)nc21)COC(C)=O]

This compound is a broad spectrum antiviral used for the treatment of herpes virus infections. This compound is commercially available or may be synthesized using a suitable preparation method, such as that disclosed in WO 87/05604 A1.

Penciclovir or 2-amino-9-[4-hydroxy-3-(hydroxymethyl)butyl]-1H-purin-6(9H)-one has the chemical structure depicted below.

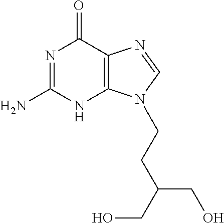 [SMILES: Nc1nc(=O)c2ncn(CCC(CO)CO)c2[nH]1]

This compound is a broad spectrum antiviral used for the treatment of herpes virus infections. This compound is commercially available or may be synthesized using a suitable preparation method, such as that disclosed in EP 0 146 516 A2.

Ganciclovir or 2-amino-9-{[(1,3-dihydroxypropan-2-yl)oxy]methyl}-6,9-dihydro-3H-purin-6-one has the chemical structure depicted below.

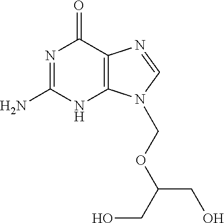 [SMILES: Nc1nc(=O)c2ncn(COC(CO)CO)c2[nH]1]

This compound is a base analogue used for the treatment of citomegalovirus infections. This compound is commercially available or may be synthesized using a suitable preparation method, such as that disclosed in U.S. Pat. No. 4,355,032.

Amantadine or (Adamantan-1-yl)amine has the chemical structure depicted below.

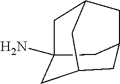 [SMILES: NC12CC3CC(CC(C3)C1)C2]

This compound is an antiviral agent used for the treatment of influenza virus infections, and it is also marketed for the treatment of movement disorders. This compound is commercially available or may be synthesized using a suitable preparation method, such as that disclosed in U.S. Pat. No. 3,152,180.

Rimantadine or 1-(1-adamant-1-yl)ethan-1-amine has the chemical structure depicted below.

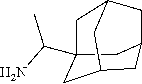

This compound is an antiviral agent used for the treatment of influenza virus infections. This compound is commercially available or may be synthesized using a suitable preparation method, such as that disclosed in FR 1.568.056 A.

Nelfinavir or (3S,4aS,8aS)-N-(1,1-Dimethylethyl)decahydro-2-[(2R,3R)-2-hydroxy-3-[(3-hydroxy-2-methylbenzoyl)amino]-4-(phenylthio)butyl]-3-isoquinolinecarboxamide has the chemical structure depicted below.

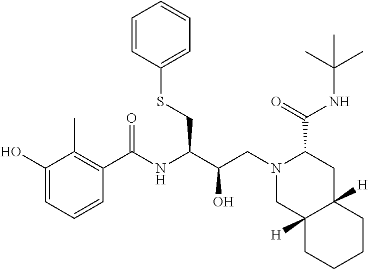 [SMILES: Cc1c(O)cccc1C(=O)N[C@@H](CSc1ccccc1)[C@H](O)CN1C[C@H]2CCCC[C@H]2C[C@H]1C(=O)NC(C)(C)C]

This compound is an HIV protease inhibitor used for the treatment of HIV infections. This compound is commercially available or may be synthesized using a suitable preparation method, such as that disclosed in WO 95/09843 A1.

Grazoprevir or Cyclopropanecarboxamide (1R,18R,20R,24S,27S)-N-{(1R,2S)-1-[(Cyclopropylsulfonyl)carbamoyl]-2-vinylcyclopropyl}-7-methoxy-24-(2-methyl-2-propanyl)-22,25-dioxo-2,21-dioxa-4,11,23,26-tetraazapentacyclo[24.2.1.03,12.05,10.0 18,20]nonacosa-3,5,7,9,11-pentaene-27-carboxamide has the chemical structure depicted below.

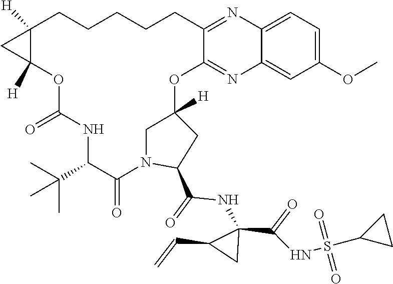 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1]

This compound is an HCV NS3/4A protease inhibitor used for the treatment of HCV infections. This compound is commercially available or may be synthesized using a suitable preparation method, such as that disclosed in WO 2010/011566 A1.

Atazanavir or methyl N-[(1S)-1-{[(2S,3S)-3-hydroxy-4-[(2S)-2-[(methoxycarbonyl)amino]-3,3-dimethyl-N-{[4-(pyridin-2-yl)phenyl]methyl}butanehydrazido]-1-phenylbutan-2-yl]carbamoyl}-2,2-dimethylpropyl]carbamate has the chemical structure depicted below.

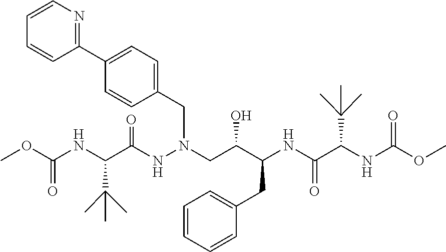

This compound is an HIV protease inhibitor used for the treatment of HIV infections. This compound is commercially available or may be synthesized using a suitable preparation method, such as that disclosed in WO 97/40029 A1.

Voxilaprevir or Cyclopropanecarboxamide, (1R,18R,20R,24S,27S,28S)-N-[(1R,2R)-2-(Difluoromethyl)-1-{[(1-methylcyclopropyl)sulfonyl]carbamoyl}cyclopropyl]-28-ethyl-13,13-difluoro-7-methoxy-24-(2-methyl-2-propanyl)-22,25-dioxo-2,21-dioxa-4,11,23,26-tetraazapentacyclo[24.2.1.03,12.05,10.018,20]nonacosa-3(12),4,6,8,10-pentaene-27-carboxamide has the chemical structure depicted below.

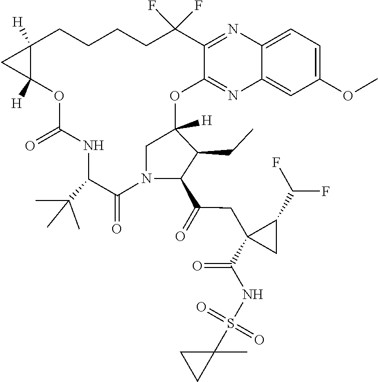 [SMILES: CC[C@@H]1[C@@H]2CN(C(=O)[C@H](C(C)(C)C)NC(=O)O[C@@H]3C[C@H]3CCCCC(F)(F)c3nc4ccc(OC)cc4nc3O2)[C@@H]1C(=O)C[C@]1(C(=O)NS(=O)(=O)C2(C)CC2)C[C@H]1C(F)F]

This compound is an HCV NS3 protease inhibitor used for the treatment of HCV infections. This compound is commercially available or may be synthesized using a suitable preparation method, such as that disclosed in WO 2014/008285 A1.

Saquinavir or (2S)-N1-[(1S,2R)-3-[(3S,4aS,8aS)-3-[[(1,1-Dimethylethyl)amino]carbonyl]octahydro-2(1H)-isoquinolinyl]-2-hydroxy-1-(phenylmethyl)propyl]-2-[(2-quinolinylcarbonyl)amino]butanediamide has the chemical structure depicted below.

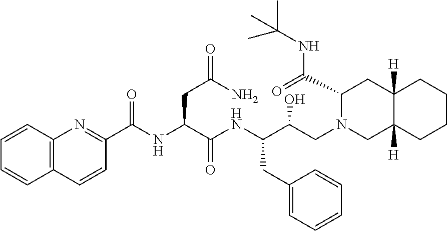 [SMILES: CC(C)(C)NC(=O)[C@@H]1C[C@@H]2CCCC[C@@H]2CN1C[C@@H](O)[C@H](Cc1ccccc1)NC(=O)[C@H](CC(N)=O)NC(=O)c1ccc2ccccc2n1]

This compound, also known as sequinavir, is an HIV protease inhibitor used for the treatment of HIV infections. This compound is commercially available or may be synthesized using a suitable preparation method, such as that disclosed in EP 0 432 695 A2.

Amprenavir or (3S)-oxolan-3-yl N-[(2S,3R)-3-hydroxy-4-[N-(2-methylpropyl)(4-aminobenzene)sulfonamido]-1-phenylbutan-2-yl]carbamate has the chemical structure depicted below.

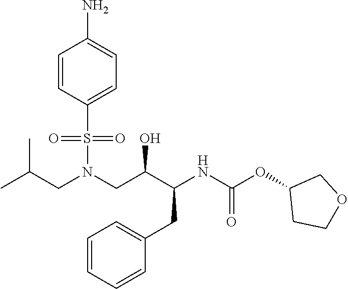 [SMILES: CC(C)CN(C[C@@H](O)[C@H](Cc1ccccc1)NC(=O)O[C@H]1CCOC1)S(=O)(=O)c1ccc(N)cc1]

This compound is an HIV protease inhibitor used for the treatment of HIV infections. This compound is commercially available or may be synthesized using a suitable preparation method, such as that disclosed in U.S. Pat. No. 5,585,397.

Indinavir or (2S)-1-[(2S,4R)-4-benzyl-2-hydroxy-4-{[(1S,2R)-2-hydroxy-2,3-dihydro-1H-inden-1-yl]carbamoyl}butyl]-N-tert-butyl-4-(pyridin-3-ylmethyl)piperazine-2-carboxamide has the chemical structure depicted below.

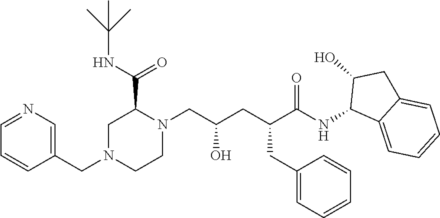 [SMILES: CC(C)(C)NC(=O)[C@@H]1CN(Cc2cccnc2)CCN1C[C@@H](O)C[C@@H](Cc1ccccc1)C(=O)N[C@H]1c2ccccc2C[C@H]1O]

This compound is an HIV protease inhibitor used for the treatment of HIV infections. This compound is commercially available or may be synthesized using a suitable preparation method, such as that disclosed in EP 0 541 168 A1.

Asunaprevir or (1R,2S)-N-[(1,1-Dimethylethoxy)carbonyl]-3-methyl-L-valyl-(4R)-4-[(7-chloro-4-methoxy-1-isoquinolinyl)oxy]-L-prolyl-1-amino-N-(cyclopropylsulfonyl)-2-ethenylcyclopropanecarboxamide has the chemical structure depicted below.

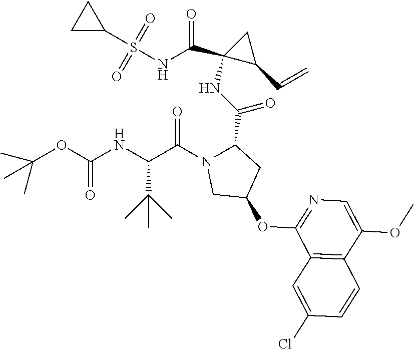 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

This compound is an HCV NS3/4A serine protease inhibitor used for the treatment of HCV infections. This compound is commercially available or may be synthesized using a suitable preparation method, such as that disclosed in WO 03/099274 A1.

Fosamprenavir or {[(2R,3S)-1-[N-(2-methylpropyl)(4-aminobenzene)sulfonamido]-3-({[(3S)-oxolan-3-yloxy]carbonyl}amino)-4-phenylbutan-2-yl]oxy}phosphonic acid has the chemical structure depicted below.

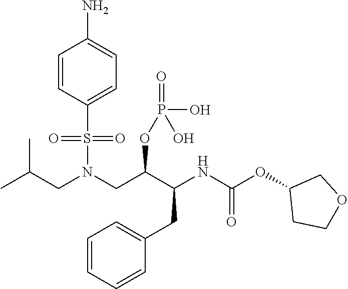 [SMILES: CC(C)CN(C[C@@H](OP(=O)(O)O)[C@H](Cc1ccccc1)NC(=O)O[C@H]1CCOC1)S(=O)(=O)c1ccc(N)cc1]

This compound is an HIV protease inhibitor used for the treatment of HIV infections. This compound is commercially available or may be synthesized using a suitable preparation method, such as that disclosed in WO 99/33815 A1.

Danoprevir or (2R,6S,12Z,13aS,14aR,16aS)-6-[(tert-Butoxycarbonyl)amino]-14a-[N-(cyclopropanesulfonyl)carbamoyl]-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 4-fluoro-1,3-dihydro-2H-isoindole-2-carboxylate has the chemical structure depicted below.

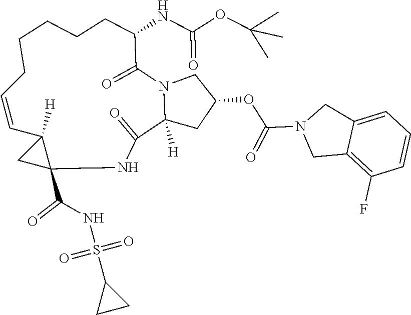 [SMILES: CC(C)(C)OC(=O)N[C@H]1CCCCC/C=C\[C@@H]2C[C@@]2(C(=O)NS(=O)(=O)C2CC2)NC(=O)[C@@H]2C[C@@H](OC(=O)N3Cc4cccc(F)c4C3)CN2C1=O]

This compound is an HCV NS3/4A protease inhibitor used for the treatment of HCV infections. This compound is commercially available or may be synthesized using a suitable preparation method, such as that disclosed in WO 2005/037214 A2.

Simeprevir or (2R,3aR,10Z,11aS,12aR,14aR)-N-(Cyclopropylsulfonyl)-2-{[2-(4-isopropyl-1,3-thiazol-2-yl)-7-methoxy-8-methyl-4-quinolinyl]oxy}-5-methyl-4,14-dioxo-2,3,3a,4,5,6,7,8,9,11a,12,13,14,14a-tetradecahydrocyclopenta[c]cyclopropa[g][1,6]diazacyclotetradecine-12a(1H)-carboxamide has the chemical structure depicted below.

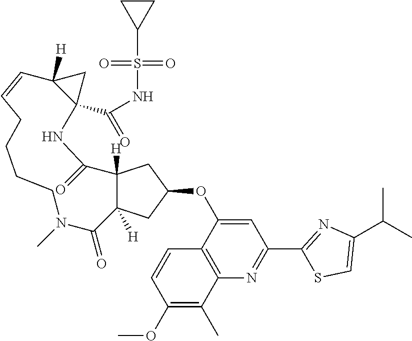 [SMILES: COc1ccc2c(O[C@@H]3C[C@H]4C(=O)N[C@]5(C(=O)NS(=O)(=O)C6CC6)C[C@H]5/C=C\CCCCN(C)C(=O)[C@@H]4C3)cc(-c3nc(C(C)C)cs3)nc2c1C]

This compound is an HCV NS3/4A protease inhibitor used for the treatment of HCV infections. This compound is commercially available or may be synthesized using a suitable preparation method, such as that disclosed in WO 2007/014926 A1.

Vaniprevir or Cyclopropanecarboxamide, (1R,21S,24S)-21-tert-Butyl-N-((1R,2R)-1-{[(cyclopropylsulfonyl)amino]carbonyl}-2-ethylcyclopropyl)-16,16-dimethyl-3,19,22-trioxo-2,18-dioxa-4,20,23-triazatetracyclo[21.2.1.14,7.06,11]-heptacosa-6,8,10-triene-24-carboxamide has the chemical structure depicted below.

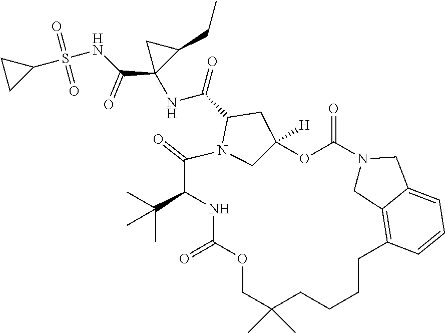

This compound, also known as MK-7009, is an HCV NS3/4A protease inhibitor used for the treatment of HCV infections. This compound is commercially available or may be synthesized using a suitable preparation method, such as that disclosed in US 2007/0027071 A1.

Narlaprevir or (1R,2S,5S)-3-[(2S)-2-[[1-(tert-butylsulfonylmethyl)cyclohexyl]carbamoylamino]-3,3-dimethylbutanoyl]-N-[(3S)-1-(cyclopropylamino)-1,2-dioxoheptan-3-yl]-6,6-dimethyl-3-azabicyclo[3.1.0]hexane-2-carboxamide has the chemical structure depicted below.

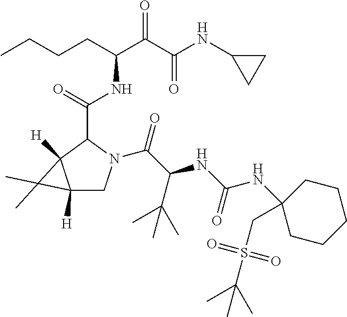 [SMILES: CCCC[C@H](NC(=O)C1[C@@H]2[C@H](CN1C(=O)[C@@H](NC(=O)NC1(CS(=O)(=O)C(C)(C)C)CCCCC1)C(C)(C)C)C2(C)C)C(=O)C(=O)NC1CC1]

This compound is an HCV NS3/4A protease inhibitor used for the treatment of HCV infections. This compound is commercially available or may be synthesized using a suitable preparation method, such as that disclosed in WO 2005/087731 A1.

Boceprevir or (1R,5S)-N-[3-Amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexane-2(S)-carboxamide has the chemical structure depicted below.

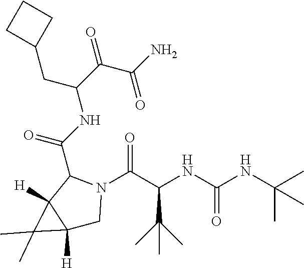 [SMILES: CC(C)(C)NC(=O)N[C@H](C(=O)N1C[C@H]2[C@@H](C1C(=O)NC(CC1CCC1)C(=O)C(N)=O)C2(C)C)C(C)(C)C]

This compound is an HCV NS3/4A protease inhibitor used for the treatment of HCV infections. This compound is commercially available or may be synthesized using a suitable preparation method, such as that disclosed in WO 02/08244 A2.

Tipranavir or N-{3-[(1R)-1-[(2R)-6-hydroxy-4-oxo-2-(2-phenylethyl)-2-propyl-3,4-dihydro-2H-pyran-5-yl]propyl]phenyl}-5-(trifluoromethyl)pyridine-2-sulfonamide has the chemical structure depicted below.

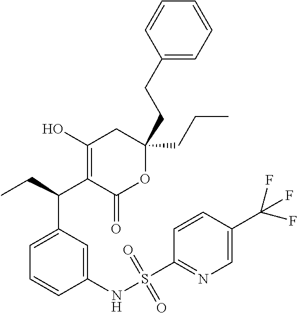 [SMILES: CCC[C@@]1(CCc2ccccc2)CC(O)=C([C@H](CC)c2cccc(NS(=O)(=O)c3ccc(C(F)(F)F)cn3)c2)C(=O)O1]

This compound is an HIV protease inhibitor used for the treatment of HIV infections. This compound is commercially available or may be synthesized using a suitable preparation method, such as that disclosed in WO 95/30670 A2.

Glecaprevir or (3aR,7S,10S,12R,21E,24aR)-7-tert-butyl-N-{(1R,2R)-2-(difluoromethyl)-1-[(1-methylcyclopropane-1-sulfonyl)carbamoyl]cyclopropyl}-20,20-difluoro-5,8-dioxo-2,3,3a,5,6,7,8,11,12,20,23,24a-dodecahydro-1H,10H-9,12-methanocyclopenta[18,19][1,10,17,3,6]trioxadiazacyclononadecino[11,12-b]quinoxaline-10-carboxamide has the chemical structure depicted below.

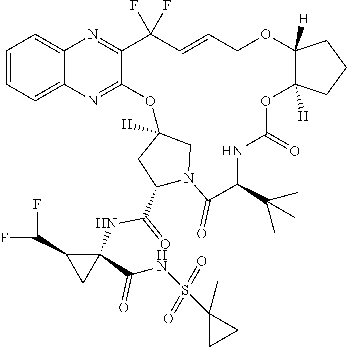 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]

This compound is an HCV NS3/4A protease inhibitor used for the treatment of HCV infections. This compound is commercially available or may be synthesized using a suitable preparation method, such as that disclosed in US 2012/0070416 A1.

Zanamivir or (2R,3R,4S)-4-guanidino-3-(prop-1-en-2-ylamino)-2-((1R,2R)-1,2,3-trihydroxypropyl)-3,4-dihydro-2H-pyran-6-carboxylic acid has the chemical structure depicted below.

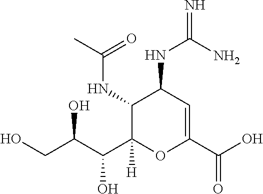 [SMILES: CC(=O)N[C@H]1[C@H]([C@H](O)[C@H](O)CO)OC(C(=O)O)=C[C@@H]1NC(=N)N]

This compound is a neuraminidase inhibitor used for the treatment of influenza virus infections. This compound is commercially available or may be synthesized using a suitable preparation method, such as that disclosed in WO 91/16320 A1.

Teicoplanin, also known as Targocid, Targosid or Teichomycin, is a glycopeptide acting as a peptidoglycan synthesis inhibitor. Teicoplanin is a complex of three main components, denominated A1, A2 and A3. Factor A2, which is present in greatest quantities in the complex extracted from Actinoplanes fermentation broth, is the factor with most significant antibacterial activity. Teicoplanin factor A2 consists in turn of a complex of 5 components, all closely related one to each other. The structure of Teicoplanin has been comprehensively described and is reported, for example, in the Merck Index, 11th edition, 1989, page 1438 , reference 9062, as well as also in the Journal of Hospital Infections, (1986) Vol. 7, supplement A, pages 79-83 (doi: 10.1016/0195-6701(86)90011-3). Teicoplanin is used as an antibiotic and has shown activity against coronavirus infections. This compound is commercially available or may be synthesized using a suitable preparation method, such as that disclosed in EP 1 671 640 A1.

Oritavancin or Vancomycin, (1S,2R,18R,19R,22S,25R,28R,40S)-48-{[(2S,3R,4S,5S,6R)-3-{[(2S,4S,5S,6S)-4-amino-5-hydroxy-4,6-dimethyloxan-2-yl]oxy}-4,5-dihydroxy-6-(hydroxymethyl)oxan-2-yl]oxy}-22-(carbamoylmethyl)-5,15-dichloro-2,18,32,35,37-pentahydroxy-19-[(2R)-4-methyl-2-(methylamino)pentanamido]-20,23,26,42,44-pentaoxo-7,13-dioxa-21,24,27,41,43-pentaazaoctacyclo[26.14.2.23,6.214,17.18,12.129,33.010,25.034,39]pentaconta-3,5,8(48),9,11,14,16,29(45),30,32,34,36,38,46,49-pentadecaene-40-carboxylic acid has the chemical structure depicted below.

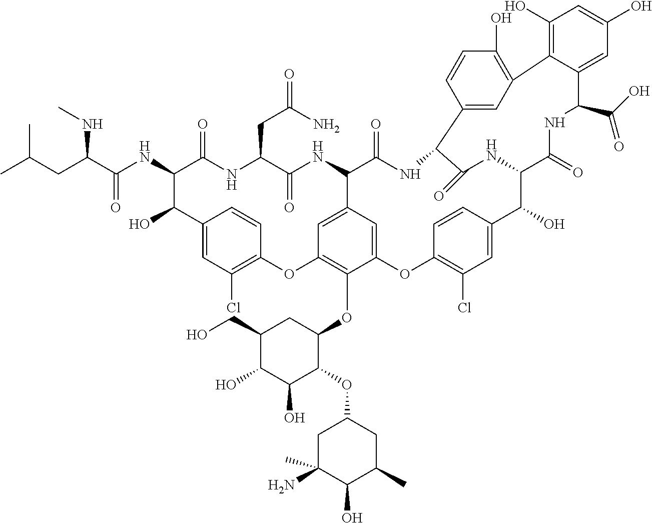

This compound is a peptidoglycan synthesis inhibitor. It is used as an antibiotic and has shown activity against coronavirus infections. This compound is commercially available or may be synthesized using a suitable preparation method, such as that disclosed in EP 0 667 353 A1.

Dalbavancin or Ristomycin A aglycone, 2-deoxy-1-O-[(3S,15R,18R,34R,35S,38S,48R,50aR)-5,31-dichloro-38-{[3-(dimethylamino)propyl]carbamoyl}-6,11,34,40,44-pentahydroxy-42-(α-D-mannopyranosyloxy)-15-(methylamino)-2,16,36,50,51,59-hexaoxo-2,3,16,17,18,19,35,36,37,38,48,49,50,50a-tetradecahydro-1H,15H,34H-20,23:30,33-dietheno-3,18:35,48-bis(iminomethano) 4,8:10,14:25,28:43,47-tetrametheno[1,14,6,22]dioxadiazacyclooctacosino[4,5-m][10,2,16]benzoxadiazacyclotetracosin-56-yl]-2-[(10-methylundecanoyl)amino]-β-D-glucopyranuronic acid has the chemical structure depicted below.

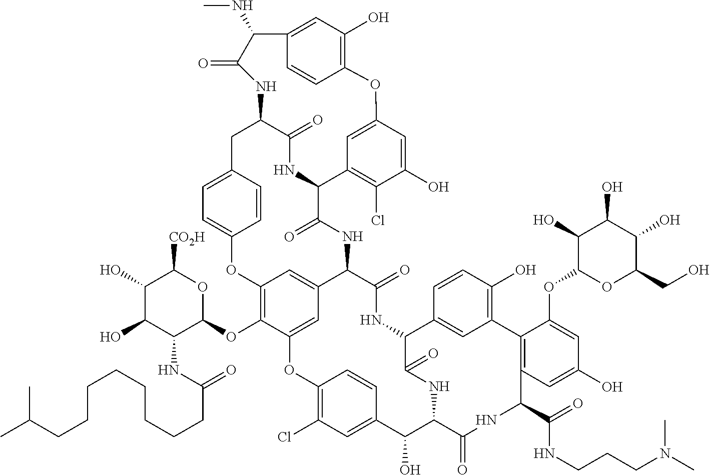

This compound is a peptidoglycan synthesis inhibitor. It is used as an antibiotic and has shown activity against coronavirus infections. This compound is commercially available or may be synthesized using a suitable preparation method, such as that disclosed in US 2005/0004050 A1.

Monensin or 4-[2-[5-Ethyl-5-[5-[6-hydroxy-6-(hydroxymethyl)-3,5-dimethyl-oxan-2-yl]-3-methyl-oxolan-2-yl]oxolan-2-yl]-9-hydroxy-2,8-dimethyl-1,6-dioxaspiro[4.5]dec-7-yl]-3-methoxy-2-methyl-pentanoic acid has the chemical structure depicted below.

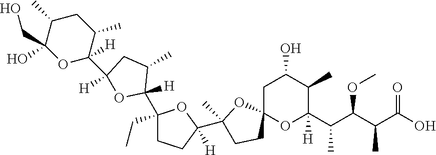 [SMILES: CC[C@@]1([C@@H]2O[C@@H]([C@H]3O[C@@](O)(CO)[C@H](C)C[C@@H]3C)C[C@@H]2C)CC[C@H]([C@]2(C)CC[C@]3(C[C@H](O)[C@@H](C)[C@@H]([C@@H](C)[C@@H](OC)[C@H](C)C(=O)O)O3)O2)O1]

This compound is a ionophore that has shown activity against coronavirus infections. This compound is commercially available or may be synthesized using a suitable preparation method, such as that disclosed in U.S. Pat. No. 3,501,568.

Mycophenolic acid or (4E)-6-(4-Hydroxy-6-methoxy-7-methyl-3-oxo-1,3-dihydro-2-benzofuran-5-yl)-4-methylhex-4-enoic acid has the chemical structure depicted below.

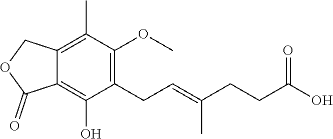 [SMILES: COc1c(C)c2c(c(O)c1C/C=C(\C)CCC(=O)O)C(=O)OC2]

This compound is an inosine-5′-monophosphate dehydrogenase inhibitor that is marketed as an immunomodulatory agent and has shown activity against coronavirus infections. This compound is commercially available or may be synthesized using a suitable preparation method, such as that disclosed in U.S. Pat. No. 4,115,197.

Gemcitabine or 4-Amino-1-(2-deoxy-2,2-difluoro-β-D-erythro-pentofuranosyl)pyrimidin-2(1H)-one has the chemical structure depicted below.

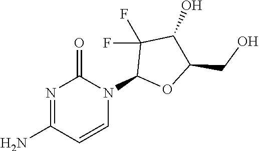 [SMILES: Nc1ccn([C@@H]2O[C@H](CO)[C@@H](O)C2(F)F)c(=O)n1]

This compound is nucleoside analogue that is used as an anticancer agent and has shown activity against coronavirus infections. This compound is commercially available or may be synthesized using a suitable preparation method, such as that disclosed in GB 2 136 425 A.

Navitoclax or 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl] propyl]amino]-3-[(trifluoromethyl)sulfonyl]phenyl]sulfonyl]benzamide has the chemical structure depicted below.

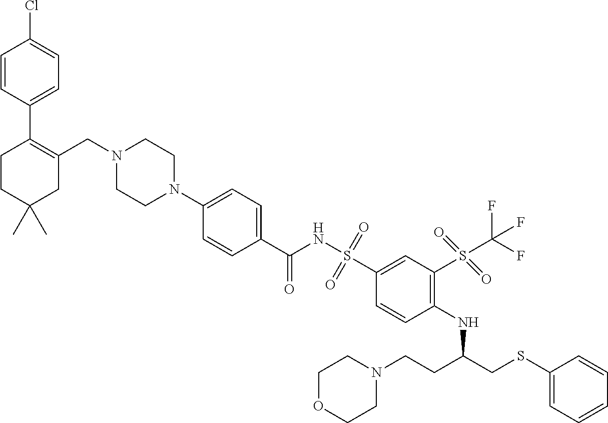 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

This compound, also known as ABT263, is a Bcl2 antagonist that is being developed as an anticancer agent and has shown activity against coronavirus infections. This compound is commercially available or may be synthesized using a suitable preparation method, such as that disclosed in US 2007/0027135 A1.

Luteolin or 2-(3,4-Dihydroxyphenyl)-5,7-dihydroxy-4H-1-benzopyran-4-one has the chemical structure depicted below.

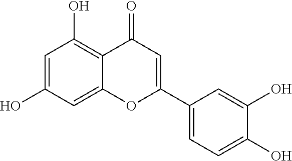 [SMILES: O=c1cc(-c2ccc(O)c(O)c2)oc2cc(O)cc(O)c12]

This compound is a natural product that has shown activity against coronavirus infections. This compound is commercially available or may be synthesized using a suitable preparation method, such as that disclosed in U.S. Pat. No. 6,538,021 B1.

Glycyrrhizin or (3β,20β)-20-Carboxy-11-oxo-30-norolean-12-en-3-yl 2-O-β-D-glucopyranuronosyl-α-D-glucopyranosiduronic acid has the chemical structure depicted below.

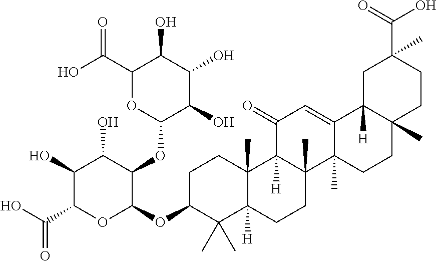

This compound is a natural product that has shown activity against coronavirus infections. This compound is commercially available or may be synthesized using a suitable preparation method, such as that disclosed in RU2082716.

Eflornithine or (RS)-2,5-diamino-2-(difluoromethyl)pentanoic acid has the chemical structure depicted below.

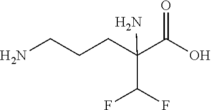 [SMILES: NCCCC(N)(C(=O)O)C(F)F]

This compound is an antiprotozoal agent that has shown activity against coronavirus infections. This compound is commercially available or may be synthesized using a suitable preparation method, such as that disclosed in GB 2 001 960 A.

Silvestrol or methyl (1R,2R,3S,3aR,8b S)-6-[[(2S,3R,6R)-6-[(1R)-1,2-dihydroxyethyl]-3-methoxy-1,4-dioxan-2-yl]oxy]-1,8b-dihydroxy-8-methoxy-3a-(4-methoxyphenyl)-3-phenyl-2,3-dihydro-1H-cyclopenta[b][1]benzofuran-2-carboxylate has the chemical structure depicted below.

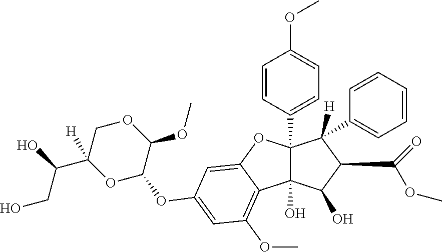 [SMILES: COC(=O)[C@H]1[C@@H](O)[C@@]2(O)c3c(OC)cc(O[C@@H]4O[C@@H]([C@H](O)CO)CO[C@H]4OC)cc3O[C@@]2(c2ccc(OC)cc2)[C@@H]1c1ccccc1]

This compound is a natural product that has shown activity against coronavirus infections. This compound is commercially available or may be synthesized using a suitable preparation method, such as that disclosed in US 2003/0181514 A1.

Emodin or 1,3,8-Trihydroxy-6-methyl anthracene-9,10-dione has the chemical structure depicted below.

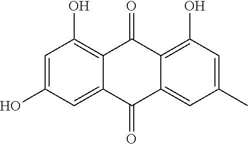 [SMILES: Cc1cc(O)c2c(c1)C(=O)c1cc(O)cc(O)c1C2=O]

This compound is a natural product that has shown activity against coronavirus infections. This compound is commercially available or may be synthesized using a suitable preparation method, such as that disclosed in CN 1546451 A.

Amiodarone or (2-{4-[(2-butyl-1-benzofuran-3-yl)carbonyl]-2,6-diiodophenoxy}ethyl)diethylamine has the chemical structure depicted below.

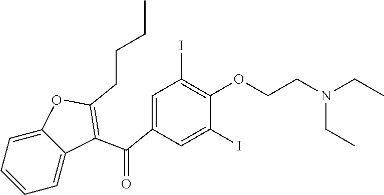 [SMILES: CCCCc1oc2ccccc2c1C(=O)c1cc(I)c(OCCN(CC)CC)c(I)c1]

This compound is a Na and K channel inhibitor that is marketed as a cardiovascular agent. It has also shown activity against coronavirus infections. This compound is commercially available or may be synthesized using a suitable preparation method, such as that disclosed in FR 2 583 754 A1.

Homoharringtonine, Omacetaxine mepesuccinate or 1-((1S,3aR,14bS)-2-Methoxy-1,5,6,8,9,14b-hexahydro-4H-cyclopenta(a)(1,3)dioxolo(4,5-h)pyrrolo(2,1-b)(3)benzazepin-1-yl) 4-methyl (2R)-2-hydroxy-2-(4-hydroxy-4-methylpentyl)butanedioate has the chemical structure depicted below.

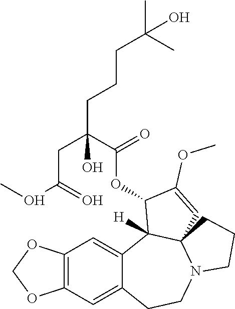 [SMILES: COC(=O)C[C@](O)(CCCC(C)(C)O)C(=O)O[C@@H]1C(OC)=C[C@]23CCCN2CCc2cc4c(cc2[C@H]13)OCO4]

This compound is a natural product that is marketed as an anticancer agent. It has also shown activity against coronavirus infections. This compound is commercially available or may be synthesized using a suitable preparation method, such as that disclosed in U.S. Pat. No. 4,152,214.

Alisporivir, also known as DEB025, Debio 025 and UNIL 025 or 8-(N-methyl-D-alanine),9-(N-ethyl-L-valine)]cyclosporine is a cyclosporine derivative with activity as cyclophilin inhibitor. It is currently in development for oncology indications and has also shown activity against coronavirus infections. This compound is commercially available or may be synthesized using a suitable preparation method, such as that disclosed in CN 103145811 A.

Hexachlorophene or 2,2′,3,3′,5,5′-Hexachloro-6,6′-dihydroxydiphenylmethane has the chemical structure depicted below.

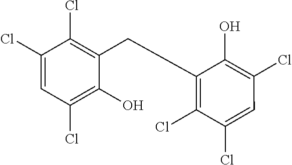 [SMILES: Oc1c(Cl)cc(Cl)c(Cl)c1Cc1c(O)c(Cl)cc(Cl)c1Cl]

This compound is a topical antiseptic that has also shown activity against coronavirus infections. This compound is commercially available or may be synthesized using a suitable preparation method, such as that disclosed in U.S. Pat. No. 2,250,480.

Camostat or N,N-Dimethylcarbamoylmethyl 4-(4-guanidinobenzoyloxy)phenylacetate has the chemical structure depicted below.

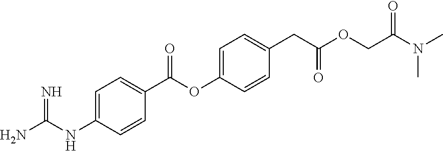 [SMILES: CN(C)C(=O)COC(=O)Cc1ccc(OC(=O)c2ccc(NC(=N)N)cc2)cc1]

This compound is a serine protease inhibitor that is marketed for gastrointestinal disorders, and its mesylate salt (camostat mesylate) is being developed for the treatment of coronavirus infections due to its actions inhibiting the transmembrane protease serine 2 (TMPRSS2), involved in the entry of the virus into host cells. This compound is commercially available or may be synthesized using a suitable preparation method, such as that disclosed in WO 2006/108643 A2.

Memantine or 3,5-dimethyltricyclo[3.3.1.13,7]decan-1amine has the chemical structure depicted below.

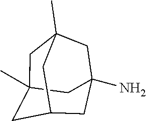

This compound is a N-methyl-D-aspartate (NMDA) receptor antagonist inhibitor that is marketed for CNS disorders, and has also shown activity against coronavirus infections. This compound is commercially available or may be synthesized using a suitable preparation method, such as that disclosed in U.S. Pat. No. 3,391,142.

Indometacin or 2-{1-[(4-Chlorophenyl)carbonyl]-5-methoxy-2-methyl-1H-indol-3-yl}acetic acid has the chemical structure depicted below.

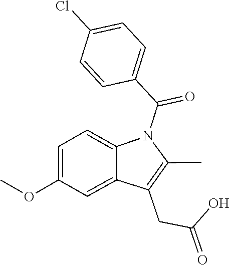 [SMILES: COc1ccc2c(c1)c(CC(=O)O)c(C)n2C(=O)c1ccc(Cl)cc1]

This compound is a COX inhibitor that is marketed for inflammatory disorders, and has also shown activity against coronavirus infections. This compound is commercially available or may be synthesized using a suitable preparation method, such as that disclosed in GB 997,638 A.

Telavancin (trade name Arbelic or Vibativ) or (1S,2R,18R,19R,22S,25R,28R,40S)-22-(2-Amino-2-oxoethyl)-5,15-dichloro-48-{[2-O-(3-{[2-(decylamino)ethyl]amino}-2,3,6-trideoxy-3-methyl-α-L-lyxo-hexopyranosyl)-β-D-glucopyranosyl]oxy}-2,18,32,35,37-pentahydroxy-19-[(N-methyl-D-leucyl)amino]-20,23,26,42,44-pentaoxo-36-{[(phosphonomethyl)amino]methyl}-7,13-dioxa-21,24,27,41,43-pentaazaoctacyclo[26.14.2.23,6.214,17.18,12.129,33.010,25.034,39]pentaconta-3,5,8(48),9,11,14,16,29(45),30,32,34,36,38,46,49-pentadecaene-40-carboxylic acid is a bactericidal lipoglycopeptide that acts as a peptidoglycan synthesis inhibitor and has the chemical structure depicted below.

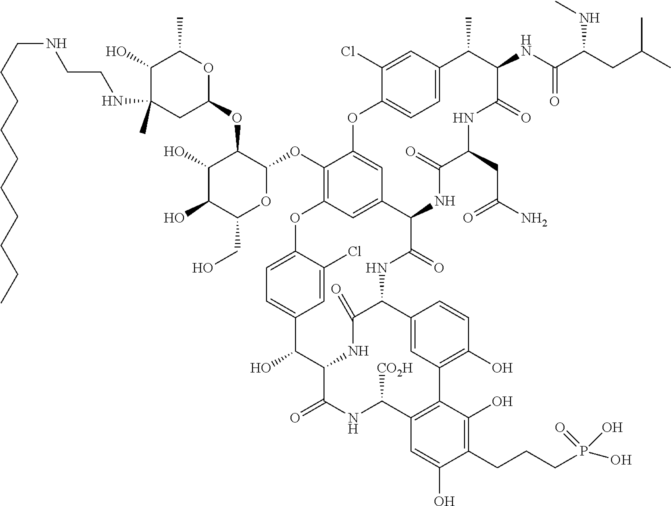 [SMILES: CCCCCCCCCCNCCN[C@@]1(C)C[C@H](O[C@H]2[C@H](Oc3c4cc5cc3Oc3ccc(cc3Cl)[C@H](C)[C@@H](NC(=O)[C@@H](CC(C)C)NC)C(=O)N[C@@H](CC(N)=O)C(=O)N[C@H]5C(=O)N[C@H]3C(=O)N[C@H](C(=O)N[C@H](C(=O)O)c5cc(O)c(CCCP(=O)(O)O)c(O)c5-c5cc3ccc5O)[C@H](O)c3ccc(c(Cl)c3)O4)O[C@H](CO)[C@@H](O)[C@@H]2O)O[C@@H](C)[C@H]1O]

This compound is marketed as an antibiotic and has also shown activity against coronavirus infections. This compound is commercially available or may be synthesized using a suitable preparation method, such as that disclosed in WO 2013/034675 A1.

Trametinib or N-[3-[3-Cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl]acetamide has the chemical structure depicted below.

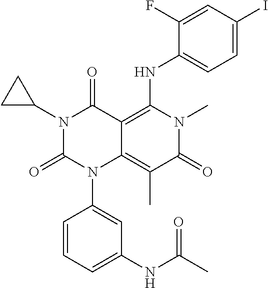 [SMILES: CC(=O)Nc1cccc(-n2c(=O)n(C3CC3)c(=O)c3c(Nc4ccc(I)cc4F)n(C)c(=O)c(C)c32)c1]

This compound is a MEK1/2 inhibitor that is marketed for oncology indications and it has also shown activity against coronavirus infections. This compound is commercially available or may be synthesized using a suitable preparation method, such as that disclosed in WO 2005/121142 A1.

N3 is a SARS-CoV-2 3CL protease inhibitor with the chemical structure depicted below.

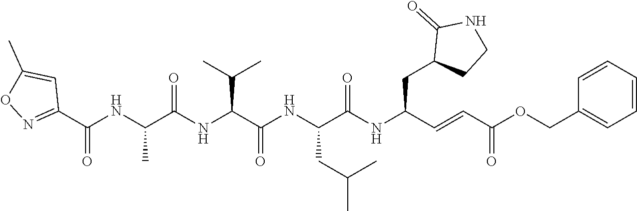 [SMILES: Cc1cc(C(=O)N[C@@H](C)C(=O)N[C@H](C(=O)N[C@@H](CC(C)C)C(=O)N[C@H](/C=C/C(=O)OCc2ccccc2)C[C@@H]2CCNC2=O)C(C)C)no1]

This drug has been described by Jin et al 2020 (https://doi.org/10.1101/2020.02.26.964882) as a SARS-CoV-2 3CL protease inhibitor also effective in cellular infection assays.

Ebselen or 2-Phenyl-1,2-benzisoselenazol-3(2H)-one has the chemical structure depicted below.

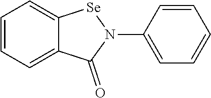 [SMILES: O=c1c2ccccc2[se]n1-c1ccccc1]

This compound is an antioxidant that is being developed for CNS disorders and it has also shown activity against coronavirus infections. This compound is commercially available or may be synthesized using a suitable preparation method, such as that disclosed in DE 38 27 093 A1.

Disulfiram or 1-(Diethylthiocarbamoyldisulfanyl)-N,N-diethyl-methanethioamide has the chemical structure depicted below.

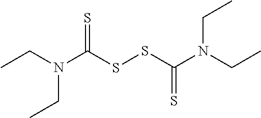 [SMILES: CCN(CC)C(=S)SSC(=S)N(CC)CC]

This compound is an aldehyde dehydrogenase inhibitor that is marketed for treating addiction, and it has also shown activity against coronavirus infections. This compound is commercially available or may be synthesized using a suitable preparation method, such as that disclosed in ES 354918 A.

Carmofur or 5-fluoro-N-hexyl-2,4-dioxo-pyrimidine-1-carboxamide has the chemical structure depicted below.

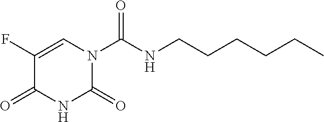 [SMILES: CCCCCCNC(=O)n1cc(F)c(=O)[nH]c1=O]

This compound is pyrimidine analogue that is marketed for oncology indications, and it has also shown activity against coronavirus infections. This compound is commercially available or may be synthesized using a suitable preparation method, such as that disclosed in ES 523519 A1.

Shikonin or 5,8-Dihydroxy-2-[(1S)-1-hydroxy-4-methylpent-3-en-1-yl]naphthalene-1,4-dione has the chemical structure depicted below.

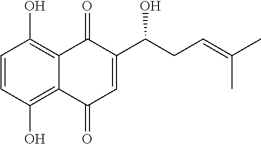 [SMILES: CC(C)=CC[C@@H](O)C1=CC(=O)c2c(O)ccc(O)c2C1=O]

This compound is a natural product that has shown activity against coronavirus infections. This compound is commercially available or may be synthesized using a suitable preparation method, such as that disclosed in JP 58-101687 A.

EIDD-1931 (β-D-N4-hydroxycytidine, NHC, 1-(2,3-Dihydroxy-4-hydroxymethyl-cyclopentyl)-4-hydroxyamino-1H-pyrimidin-2-one) and its orally available prodrug EIDD-2801 (isobutyric acid 3,4-dihydroxy-5-(4-hydroxyamino-2-oxo-2H-pyrimidin-1-yl)-tetrahydro-furan-2-ylmethyl ester) are nucleoside analogues with the chemical structures depicted below

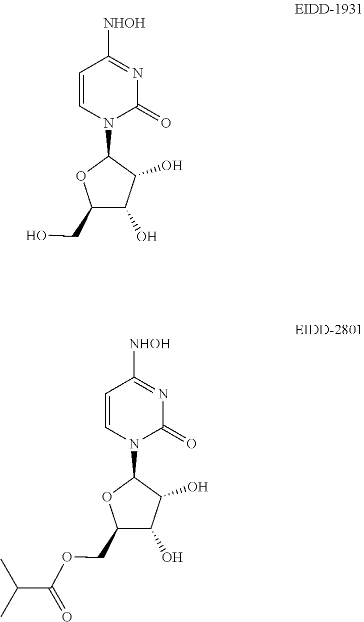

They are being investigated for the treatment of COVID-19 as disclosed by Sheahan et al 2020 (https://doi.org/10.1101/2020.03.19.997890).

Ivermectin (CAS 70288-86-7) is a mixture of two closely related macrocyclic lactones derived from Streptomyces avermitilis with the chemical structures depicted below.

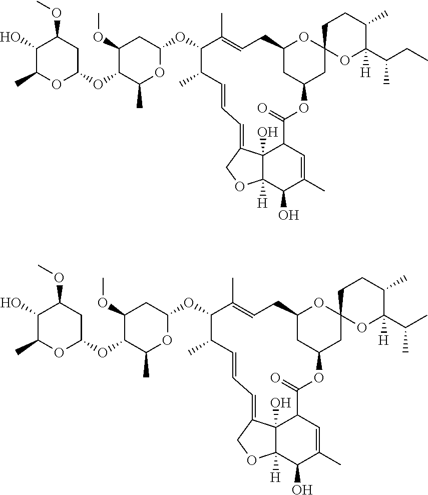

It has antiparasitic activity and is being investigated as a COVID-19 treatment. This compound is commercially available or may be synthesized using a suitable preparation method, such as that disclosed in U.S. Pat. No. 6,265,571 B1.

Tideglusib or 2-(1-Naphthalenyl)-4-(phenylmethyl)-1,2,4-thiadiazolidine-3,5-dione has the chemical structure depicted below.

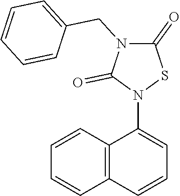 [SMILES: O=c1sn(-c2cccc3ccccc23)c(=O)n1Cc1ccccc1]

This compound is a GSK3beta inhibitor currently in development for myotonic dystrophy and CNS indications. It has also shown activity against coronavirus infections. This compound is commercially available or may be synthesized using a suitable preparation method, such as that disclosed in US 2005/0222220 A1.

PX-12 or 2-sec-butyldisulfanyl-1H-imidazole is a thioredoxin-1 inhibitor with the chemical structures depicted below.

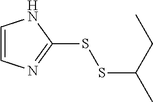

It was in development for oncology indications. It has been identified as a SARS-Cov-2 3 CL protease inhibitor by Jin et al 2020 (https://doi.org/10.1101/2020.02.26.964882).

Hydrocortisone or (11β)-11,17,21-Trihydroxypregn-4-ene-3,20-dione has the chemical structure depicted below.

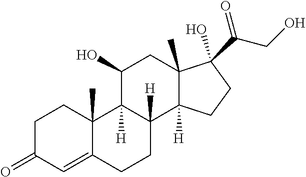

This compound is a glucocorticoid receptor agonist used as an immunomodulatory agent. It has been used to treat complications of coronavirus infections. This compound is commercially available or may be synthesized using a suitable preparation method, such as that disclosed in U.S. Pat. No. 2,658,023.

Methylprednisolone or (1S,2R,8S,10S,11S,14R,15S,17S)-14,17-dihydroxy-14-(2-hydroxyacetyl)-2,8,15-trimethyltetracyclo[8.7.0.02,7.011,15]heptadeca-3,6-dien-5-one has the chemical structure depicted below.

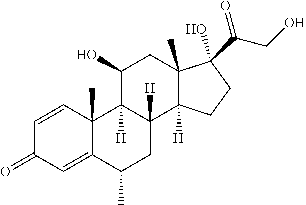

This compound is a glucocorticoid receptor agonist used as an immunomodulatory agent. It has been used to treat complications of coronavirus infections. This compound is commercially available or may be synthesized using a suitable preparation method, such as that disclosed in BE 612.700.

Ciclesonide or 2-[(1S,2S,4R,8S,9S,11S,12S,13R)-6-cyclohexyl-11-hydroxy-9,13-dimethyl-16-oxo-5,7-dioxapentacyclo[10.8.0.0^{2,9}.0^{4,8}.0^{13,18}]icosa-14,17-dien-8-yl]-2-oxoethyl-2-methylpropanoate has the chemical structure depicted below.

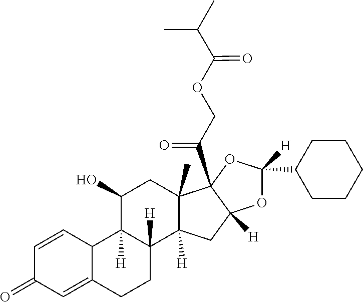

This compound is a glucocorticoid receptor agonist used as an immunomodulatory agent. It has been used to treat complications of coronavirus infections. This compound is commercially available or may be synthesized using a suitable preparation method, such as that disclosed in GB 2 247 680 A.

Naproxen or (+)-(S)-2-(6-Methoxynaphthalen-2-yl)propanoic acid has the chemical structure depicted below.

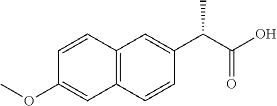 [SMILES: COc1ccc2cc([C@H](C)C(=O)O)ccc2c1]

This compound is a COX inhibitor used as anti-inflammatory agent. It has been used to treat complications of coronavirus infections. This compound is commercially available or may be synthesized using a suitable preparation method, such as that disclosed in U.S. Pat. No. 3,652,683.

Peginterferon alpha-2b is a PEGylated form of recombinant interferon alpha-2b used as immunomodulatory agent and for the treatment of viral infections. It is marketed as Pegintron, Pegetron. Pegatron, Cylatron, Sylatron, Cimipeg, Vipeg, Preferon, Viraferon Peg and Paigbin, among other brand names. This compound is commercially available or may be synthesized using a suitable preparation method, such as that disclosed in WO 2009/030066 A1.

Colchicine or (S)—N-(1,2,3,10-Tetramethoxy-9-oxo-5,6,7,9-tetrahydro-benzo[a]heptalen-7-yl)acetamide has the chemical structure depicted below.

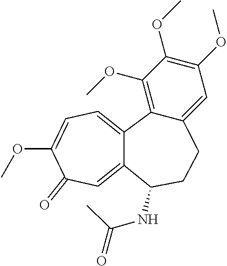 [SMILES: COc1cc2c(c(OC)c1OC)-c1ccc(OC)c(=O)cc1[C@@H](NC(C)=O)CC2]

This compound inhibits tubulin polymerization and is used as immunomodulatory agent. It has been used to treat complications of coronavirus infections. This compound is commercially available or may be synthesized using a suitable preparation method, such as that disclosed in GB 923,421.

Opaganib or 3-(4-Chlorophenyl)-N-(4-pyridinylmethyl)tricyclo[3.3.1.13,7] decane-1-carboxamide has the chemical structure depicted below.

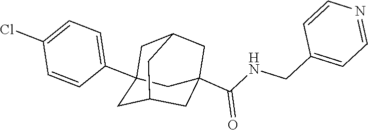 [SMILES: O=C(NCc1ccncc1)C12CC3CC(C1)CC(c1ccc(Cl)cc1)(C3)C2]

This compound, also known as ABC 294640, is a sphingosine kinase-2 (SK2) selective inhibitor that is currently developed for oncology indications. It has shown activity against coronavirus infections. This compound is commercially available or may be synthesized using a suitable preparation method, such as that disclosed in US 2006/0287317 A1.

Novaferon is a recombinant interferon alpha-2b used as immunomodulatory agent and for the treatment of viral infections. It is marketed as Novaferon by Genova Biotech Co.

SNG001 is an inhaled formulation of interferon beta-1a currently in development for the treatment of COVID-19 by Synairgen.

Peginterferon lambda is a PEGylated form of interferon lambda-1a currently in clinical development for the treatment of hepatitis virus infections and COVID-19. This drug is developed by Eiger Biopharmaceuticals Inc.

Rebif is a recombinant interferon beta-1a marketed by Merck KGaA as an immunomodulatory agent. It is also in clinical development for the treatment of viral infections such as HIV and SARS-CoV-2.

Sargramostim is a recombinant granulocyte macrophage colony-stimulating factor marketed as Leukine by Immunex Corp for the treatment of neutropenia.

Anakinra is a recombinant interleukin 1 (IL-1) receptor antagonist protein with some modifications. It is marketed as Kineret for the treatment of inflammatory diseases and it is being studied for the treatment of COVID-19 complications.

Baricitinib or 2-[1-Ethylsulfonyl-3-[4-(7H-pyrrolo[2,3-d]pyrimidin-4-yl)pyrazol-1-yl]azetidin-3-yl]acetonitrile has the chemical structure depicted below.

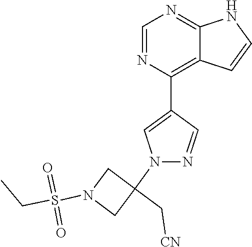 [SMILES: CCS(=O)(=O)N1CC(CC#N)(n2cc(-c3ncnc4[nH]ccc34)cn2)C1]

This compound is a JAK1/2 inhibitor that is currently marketed for the treatment of inflammatory diseases such as rheumatoid arthritis. It is also developed for the treatment of COVID-19 complications. This compound is commercially available or may be synthesized using a suitable preparation method, such as that disclosed in WO 2009/114512 A1.

Antibodies from recovered COVID-19 patients are being used for the treatment of COVID-19 in clinical trials. The objective of this treatment is to provide infected patients with neutralizing antibodies produced by recovered patients.

Bevacizumab is a monoclonal antibody directed against vascular endothelial growth factor A (VEGF-A). It is marketed by F. Hoffmann-La Roche Ltd as Avastin for the treatment of oncology indications owing to its antiangiogenic activity, and it is currently developed for the treatment of complications of COVID-19.

PD-1 antibody is a monoclonal antibody against the Programmed Death receptor 1 used as an immunomodulatory agent for the treatment of oncology indications. It is being developed for the treatment of complications of COVID-19.

Leronlimab is a monoclonal antibody against CCRS, currently developed by Cytodyn Inc as an immunomodulatory agent and for the treatment of HIV infections. It is also developed for the treatment of complications of COVID-19.

Camrelizumab is a monoclonal antibody against PD-1 that is marketed as AiRuiKa for B-Cell Hodgkin Lymphoma by Jiangsu Hengrui Medicine Co Ltd. It is being developed for the treatment of complications of COVID-19.

Sarilumab is a monoclonal antibody against interleukin-6 (IL6) Receptor, acting as an IL6 antagonist and marketed by Sanofi-Aventis as Kevzara for the treatment of rheumatoid arthritis. It is being developed for the treatment of complications of COVID-19.

Tocilizumab is a monoclonal antibody against IL6 Receptor, acting as an IL6 antagonist and marketed by Roche as Actemra for the treatment of rheumatoid arthritis and related diseases. It is being developed for the treatment of complications of COVID-19.

Gimsilumab is a monoclonal antibody against human granulocyte-macrophage colony-stimulating factor currently in clinical development for ankylosing spondylitis. It is also being developed for the treatment of complications of COVID-19.

TJM2 (also known as TJ003234) is a monoclonal antibody against the receptor of granulocyte-macrophage colony-stimulating factor, currently in clinical development for rheumatoid arthritis. It is also being developed for the treatment of complications of COVID-19.

Lenzilumab is a monoclonal antibody against the receptor of granulocyte-macrophage colony-stimulating factor, currently in clinical development for lymphoma. It is also being developed for the treatment of complications of COVID-19.

Siltuximab is an anti-IL-6 chimeric monoclonal antibody currently marketed as Sylvant for the treatment of Giant Lymph Node Hyperplasia (Castleman's Disease). It is being developed for the treatment of complications of COVID-19.

Eculizumab is a monoclonal antibody against the complement protein C5 marketed as Soliris for the treatment of atypical haemolytic uremic syndrome. It is being developed for the treatment of complications of COVID-19.

Mavrilimumab is a monoclonal antibody against the receptor of granulocyte-macrophage colony-stimulating factor, currently in clinical development for autoimmune disorders. It is also being developed for the treatment of complications of COVID-19.

Canakinumab is a monoclonal antibody against human interleukin-1beta (IL-1 Beta) marketed as Ilaris for the treatment of cryopyrin associated periodic syndromes. It is also being developed for the treatment of complications of COVID-19.

Namilumab is a monoclonal antibody against the receptor of granulocyte-macrophage colony-stimulating factor, currently in clinical development for ankylosing spondylitis. It is also being developed for the treatment of complications of COVID-19.

Emapalumab is a monoclonal antibody against interferon gamma marketed as immunosuppressant for the treatment of hemophagocytic lymphohistiocytosis. It is also being developed for the treatment of complications of COVID-19.

Meplazumab is a monoclonal antibody against CD147 (basigin) that is currently in clinical trials for the treatment of COVID-19.

Ruxolitinib or (3R)-3-Cyclopentyl-3-[4-(7H-pyrrolo[2,3-d]pyrimidin-4-yl)-1H-pyrazol-1-yl]propanenitrile has the chemical structure depicted below.

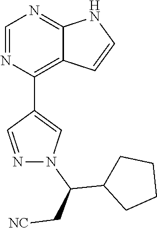 [SMILES: N#CC[C@H](C1CCCC1)n1cc(-c2ncnc3[nH]ccc23)cn1]

This compound is a JAK1/2 inhibitor that is currently marketed for the treatment of haematological malignancies. It is also developed for the treatment of COVID-19. This compound is commercially available or may be synthesized using a suitable preparation method, such as that disclosed in US 2007/0135461 A1.

Fingolimod or 2-Amino-2-[2-(4-octylphenyl)ethyl] propane-1,3-diol has the chemical structure depicted below.

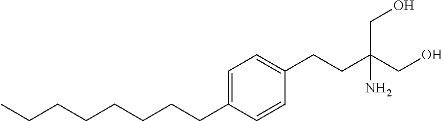 [SMILES: CCCCCCCCc1ccc(CCC(N)(CO)CO)cc1]

This compound is a Sphingosine-1-phosphate Receptor modulator that is currently marketed for the treatment of multiple sclerosis as an immunomodulatory agent. It is also developed for the treatment of COVID-19. This compound is commercially available or may be synthesized using a suitable preparation method, such as that disclosed in EP 0 627 406 A1.

Ifenprodil or 1-Methyl-2-hydroxy-2-(4-hydroxyphenyl)ethyl-1-(4-benzylpiperidine) has the chemical structure depicted below.

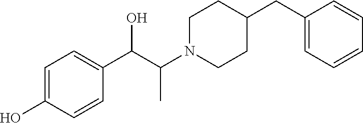 [SMILES: CC(C(O)c1ccc(O)cc1)N1CCC(Cc2ccccc2)CC1]

This compound is a NDMA Glutamate receptor antagonist (Glu2NB) that is currently in development for idiopathic pulmonary fibrosis. It is also developed for the treatment of COVID-19. This compound is commercially available or may be synthesized using a suitable preparation method, such as that disclosed in GB 1,159,449 A.

APN01 is a recombinant, highly glycosylated form of human angiotensin converting enzyme 2 (rhACE2) that is currently developed by Apeiron Biologics for acute respiratory distress syndrome, pulmonary arterial hypertension and COVID-19.

Brilacidin or N4,N6-bis[3-[[5-[(aminoiminomethyl)amino]-1-oxopentyl]amino]-2-[(3R)-3-pyrrolidinyloxy]-5-(trifluoromethyl)phenyl]-4,6-pyrimidinedicarboxamide has the chemical structure depicted below.

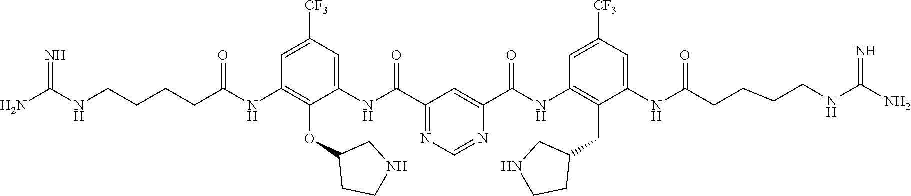

This compound is defensin mimetic that targets bacterial membranes and that is currently in development as an antibacterial agent. It is also developed for the treatment of COVID-19. This compound is commercially available or may be synthesized using a suitable preparation method, such as that disclosed in WO 2010/062573 A1.

BXT-25 is an hemoglobin based glycopolymer that is currently developed by Bioxytran Inc for hypoxia and acute respiratory distress syndrome. It is also being developed for the treatment of COVID-19 complications.

Nafamostat or 6-[amino(imino)methyl]-2-naphthyl 4-{[amino(imino)methyl]amino}benzoate has the chemical structure depicted below.

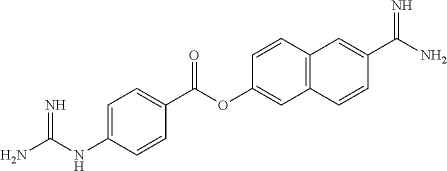 [SMILES: N=C(N)Nc1ccc(C(=O)Oc2ccc3cc(C(=N)N)ccc3c2)cc1]

This compound is a serine protease inhibitor that inhibits the complement C1s protease. It is marketed for the treatment of pancreatitis and it is also developed for the treatment of COVID-19. This compound is commercially available or may be synthesized using a suitable preparation method, such as that disclosed in EP 0 048 433 A2.

Losartan or 1-[1-[[2′-(2H-Tetrazol-5-yl)biphenyl-4-yl]methyl]-2-butyl-4-chloro-1H-imidazol-5-yl]methanol has the chemical structure depicted below.

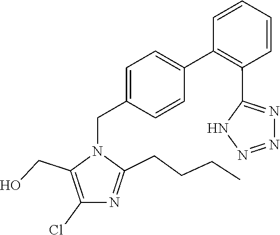 [SMILES: CCCCc1nc(Cl)c(CO)n1Cc1ccc(-c2ccccc2-c2nnn[nH]2)cc1]

This compound is Type 1 Angiotensin II Receptor inhibitor that is marketed for the treatment of hypertension and it is also developed for the treatment of COVID-19. This compound is commercially available or may be synthesized using a suitable preparation method, such as that disclosed in WO 2010/029457 A2.

Alteplase is a recombinant Tissue Plasminogen Activator, a serine protease enzyme that converts plasminogen to plasmin. It is marketed by F. Hoffmann-La Roche Ltd as Actilyse, Activacin, Grtpa, Cathflo, Cathflo Activase and Lysatec for the treatment of thrombosis, myocardial infarctions and other cardiovascular disorders. It is being developed for the treatment of COVID-19 complications.

AT-001 or 2-(8-oxo-7-{[5-(trifluoromethyl)-1,3-benzothiazol-2-yl]methyl}-7H,8H-pyrazino[2,3-d]pyridazin-5-yl)acetic acid has the chemical structure depicted below.

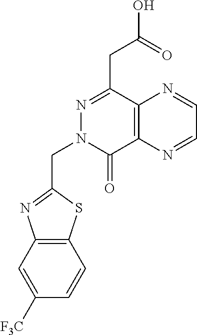 [SMILES: O=C(O)Cc1nn(Cc2nc3cc(C(F)(F)F)ccc3s2)c(=O)c2nccnc12]

This compound is an aldose reductase inhibitor under development by Applied Therapeutics for the treatment of diabetic complications including type 2 diabetes, diabetic cardiomyopathy, diabetic peripheral neuropathy, acute myocardial infarction (liquid formulation), acute lung inflammation and cardiomyopathy in critical COVID-19 infections.

Plitidepsin or (S)—N—((R)-1-(((3S,6R,7S,10R,11S,15S,17S,20S,25aS)-10-((S)-sec-butyl)-11-hydroxy-20-isobutyl-15-isopropyl-3-(4-methoxybenzyl)-2,6,17-trimethyl-1,4,8,13,16,18,21-heptaoxodocosahydro-1H-pyrrolo[2,1-f][1,15,4,7,10,20]dioxatetraazacyclotricosin-7-yl)amino)-4-methyl-1-oxopentan-2-yl)-N-methyl-1-(2-oxopropanoyl)pyrrolidine-2-carboxamide has the chemical structure depicted below.

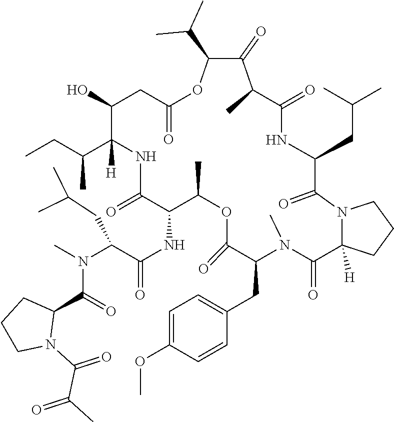 [SMILES: CC[C@H](C)[C@H]1NC(=O)[C@@H](NC(=O)[C@@H](CC(C)C)N(C)C(=O)[C@@H]2CCCN2C(=O)C(C)=O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O]

This compound inhibits elongation factor 1 alpha 2 thereby blocking protein synthesis and it is marketed as Aplidin for the treatment of multiple myeloma. It is also developed for the treatment of COVID-19. This compound is commercially available or may be synthesized using a suitable preparation method, such as that disclosed in U.S. Pat. No. 5,294,603.

Dipyridamole or 2,2′,2″,2′″-((4,8-di(piperidin-1-yl)pyrimido[5,4-d]pyrimidine-2,6-diyl)bis(azanetriyl))tetraethanol has the chemical structure depicted below.

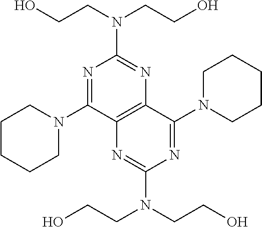 [SMILES: OCCN(CCO)c1nc(N2CCCCC2)c2nc(N(CCO)CCO)nc(N3CCCCC3)c2n1]

This compound is a phosphodiesterase inhibitor with anticoagulant actions. It is marketed for the treatment of thromboembolism. It is also developed for the treatment of complications of COVID-19. This compound is commercially available or may be synthesized using a suitable preparation method, such as that disclosed in GB 807,826.

Ebastine or 1-[4-(1,1-Dimethylethyl)phenyl]-4-[4-(diphenylmethoxy)-1-piperidinyl]-1-butanone with the chemical structure depicted below.

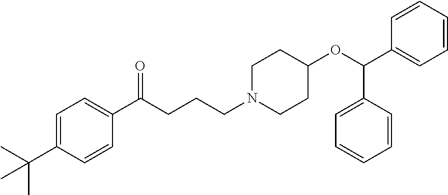 [SMILES: CC(C)(C)c1ccc(C(=O)CCCN2CCC(OC(c3ccccc3)c3ccccc3)CC2)cc1]

This compound is a H1 Receptor antagonist marketed for the treatment of allergy. It is also developed for the treatment of COVID-19. This compound is commercially available or may be synthesized using a suitable preparation method, such as that disclosed in EP 0 134 124 A1.

Azithromycin or (2R,3S,4R,5R,8R,10R,11R,12S,13S,14R)-13-[(2,6-Dideoxy-3-C-methyl-3-O-methyl-α-L-ribo-hexopyranosyl)oxy]-2-ethyl-3,4,10-trihydroxy-3,5,6,8,10,12,14-heptamethyl-11-[[3,4,6-trideoxy-3-(dimethylamino)-β-D-xylo-hexopyranosyl]oxy]-1-oxa-6-azacyclopentadecan-15-one has the chemical structure depicted below.

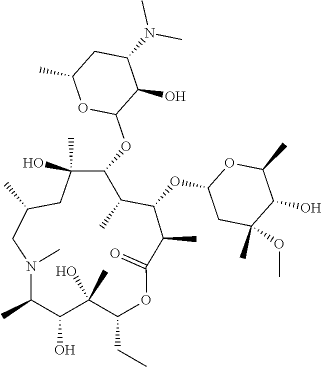 [SMILES: CC[C@H]1OC(=O)[C@H](C)[C@@H](O[C@H]2C[C@@](C)(OC)[C@@H](O)[C@H](C)O2)[C@H](C)[C@@H](OC2O[C@H](C)C[C@H](N(C)C)[C@H]2O)[C@](C)(O)C[C@@H](C)CN(C)[C@H](C)[C@@H](O)[C@]1(C)O]

This compound is an antibiotic that inhibits translation of bacterial mRNA by binding to the 50S subunit of the bacterial ribosome. It is marketed for the treatment of bacterial infections and it is also being developed for the treatment of COVID-19. This compound is commercially available or may be synthesized using a suitable preparation method, such as that disclosed in BE 892.357.

Solnatide (AP-301) or L-Cysteine, L-cysteinylglycyl-L-glutaminyl-L-arginyl-L-α-glutamyl-L-threonyl-L-prolyl-L-α-glutamylglycyl-L-alanyl-L-α-glutamyl-L-alanyl-L-lysyl-L-prolyl-L-tryptophyl-L-tyrosyl-, cyclic (1→17)-disulfide is a cyclic peptide that activates the pulmonary epithelial sodium channel (ENaC). It has the chemical structure depicted below.

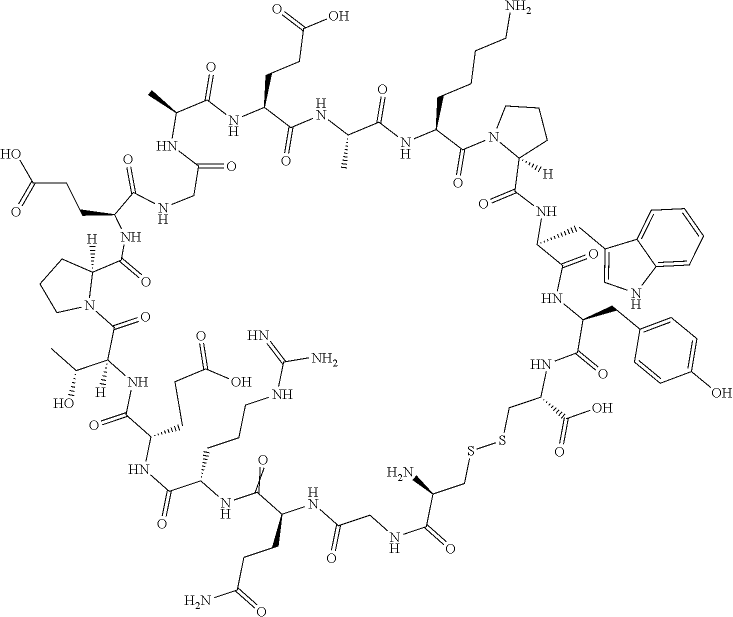 [SMILES: C[C@@H]1NC(=O)CNC(=O)[C@H](CCC(=O)O)NC(=O)[C@@H]2CCCN2C(=O)[C@H]([C@@H](C)O)NC(=O)[C@H](CCC(=O)O)NC(=O)[C@H](CCCNC(=N)N)NC(=O)[C@H](CCC(N)=O)NC(=O)CNC(=O)[C@@H](N)CSSC[C@@H](C(=O)O)NC(=O)[C@H](Cc2ccc(O)cc2)NC(=O)[C@H](Cc2c[nH]c3ccccc23)NC(=O)[C@@H]2CCCN2C(=O)[C@H](CCCCN)NC(=O)[C@H](C)NC(=O)[C@H](CCC(=O)O)NC1=O]

It is in clinical development for pneumonia and other pulmonary affections, including influenza. It is also in development for the treatment of complications of COVID-19. This compound is commercially available or may be synthesized using a suitable preparation method, such as that disclosed in WO 2014/173843 A1.

Selinexor or (2Z)-3-{3-[3,5-Bis(trifluoromethyl)phenyl]-1,2,4-triazol-1-yl}-N′-pyrazin-2-ylprop-2-enehydrazide has the chemical structure depicted below.

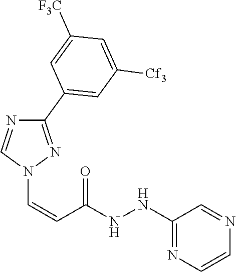

This compound is a selective inhibitor of nuclear export that is marketed for the oncology indications. It is also being developed for the treatment of COVID-19. This compound is commercially available or may be synthesized using a suitable preparation method, such as that disclosed in WO 2013/019548 A1.

In particular, the combinations for use according to the invention are administered as pharmaceutical compositions, which comprise the corresponding (active) compounds and a pharmaceutically acceptable excipient, as previously defined.

The combinations for use according to the invention will typically be administered once or more times a day, for example 1, 2, 3 or 4 times daily, with typical total daily doses depending on the particular compound and severity of the disease, and may be easily determined by the skilled practitioner.

In another particular embodiment, the viral infection is an infection by SARS-CoV-2 virus.

The following examples represent specific embodiments of the present invention. They do not intend to limit in any way the scope of the invention defined in the present description.

### EXAMPLES

### Materials and Methods

### SARS-CoV2

### Example 1

### Protein Expression and Purification of the SARS-CoV-2 3CL Protease

The coding sequence of SARS-CoV-2 3C-like proteinase (NCBI Ref. seq. YP_009725301.1 dated 30 Mar. 2020) was synthesized chemically by Bioneer (Daejeon, Korea) and cloned into a bacteriophage T7-based expression vector. The plasmid DNA was transformed into E. coli BL21 (DE3) for protein expression. E. coli BL21 (DE3) cells were grown on Luria-Bertani (LB) agar plates containing 150 μg/ml ampicillin. Several colonies were picked and grown in capped test tubes with 10 ml LB broth containing 150 μg/ml ampicillin. A cell stock composed of 0.85 ml culture and 0.15 ml glycerol was prepared and frozen at −80° C. for use in a large culture.

The frozen cell stock was grown in 5 ml LB medium and diluted into 1000 ml fresh LB medium. The culture was incubated at 37° C. with shaking until an OD600 of 0.6-0.8 was reached. At this point, the expression of SARS-CoV-2 3CLpro was induced using isopropyl-β-D-1-thiogalactopyranoside (IPTG) at a final concentration of 1 mM. The culture was further grown at 37° C. for 3 h in a shaking incubator.

Cells were harvested by centrifugation at 7650 g (6500 rev/min) for 10 min in a high-speed refrigerated centrifuge at 4° C. The cultured cell paste was resuspended in 25 ml of a buffer consisting of 20 mM Tris pH 7.5, 1 mM phenylmethylsulfonyl fluoride (PMSF), 10 μg/ml DNase I. The cell suspension was disrupted using an ultrasonic cell disruptor (Digital Sonifier 450, Branson, USA). Cell debris was pelleted by centrifugation at 24 900 g (15 000 rev/min) for 30 min in a high-speed refrigerated ultra-centrifuge at 4° C.

The protein was purified by cation chromatography using a 5 ml Hi-Trap Q column (GE Healthcare, Piscataway, N.J., USA). The column was equilibrated with a buffer consisting of 20 mM Tris pH 7.5 and the pooled fractions were loaded. The column was eluted using a linear NaCl gradient to 1.0 M NaCl and the protein was eluted at 0.18 M NaCl.

### Example 2

### Docking Methods

As a preliminary step in the identification of compounds which may have the capacity of inhibiting protein 3CLpro of SARS-CoV-2, an in-silico docking method was applied to 300 compounds from a library of clinically used drugs. The method allowed to identify a subset of compounds that were able to form a covalent bond with Cys145 through a Michael addition reaction.

We first proceeded with the validation of the docking workflow, by performing covalent redocking of the reference compound N3. The redocked pose was comparable to that of the crystal, with a root mean square deviation (RMSD) of 1.33 Å calculated over the maximum common structure of the ligands and with a docking score (an approximation of the binding free energy ΔGbind), of −9.82 kcal/mol. We then applied the same workflow to the molecules of the library. Several molecules could form a covalent bond with Cys145, through a Michael Addition reaction. Then, in order to identify potential non-covalent inhibitors, we set out to perform non-covalent docking with the remaining compounds. We first validated the non-covalent docking workflow by redocking the ligand N3; a pose similar to the crystallized compound N3 was obtained, with an RMSD of 1.17 Å over the maximum common structure of the ligands, and with a non-covalent redocking score of −11.31 kcal/mol. Finally, we applied the same workflow to the remaining compounds that could not form a covalent bond with Cys145.

### Protein Preparation

The protein crystal structure of SARS-CoV-2 3CLpro with PDB code 6LU7 was used. The protein was prepared for docking studies through the Protein Wizard tool (Schrödinger). Missing side chains and loops were modelled with the in-built functionality using Prime (Schrödinger). Water molecules beyond 3.0 Å from heteroatoms, or with less than 3 bonds with non-waters after minimization, were removed; protein residue protonation states were assigned with PROPKA (Schrödinger) at pH 7.0. Finally, protein heavy atoms were minimized to convergence to 0.30 Å RMSD, using the OPLS_2005 force field (Schrödinger).

### Ligand Preparation

Ligands were prepared with the LigPrep (Schrödinger) at pH 7.0±1.0 using Epik (Schrödinger), including the options for desalting and for tautomer generation. Computation of stereoisomers was performed by retaining specified chiralities.

### Docking Studies

### Covalent Redocking

First, the protein structure was edited by removing the covalent bond between the compound N3 and the thiol group of Cys145 of the SARS-CoV-2 3CLpro. Then, the unbound compound N3 was prepared with LigPrep at pH 7.0±0.5 using Epik prior to redocking. Next, we set out to redock the reference compound N3 in the binding site to validate the covalent docking workflow, performed with the CovDock covalent docking tool (Schrödinger). We selected Cys145 as the reactive residue, and the original pose of compound N3 was used to define the centroid for the grid box center. Michael Addition was chosen as reaction type for the prepared compound N3. Covalent redocking was performed with an energy cutoff of 2.5 kcal/mol to retain poses for further refinement, up to a maximum of 200 poses.

### Non-Covalent Redocking

A non-covalent redocking procedure for compound N3 was also performed using Glide (Schrödinger) to identify non-covalent inhibitors. Grid dimensions were 10 Å×10 Å×10 Å for the inner box, and 35 Å×35 Å×35 Å for the outer box, using the original ligand pose to define the box centroid. The Van der Waals radius scaling was set to 1.00 to reproduce the binding pose. Standard Precision (SP) settings were selected, including flexible ligand, Nitrogen inversions, and ring conformations sampling, with the addition of Epik state penalties to the docking scores.

### Docking of the Top-Scoring 300 Compounds

We performed docking on 300 compounds from a library of clinically used drugs and identified a subset of compounds that were able to form a covalent bond with Cys145 through a Michael addition reaction. We performed the docking calculations through the same workflow used for covalent redocking. Similarly, to identify non-covalent inhibitors among the remaining compounds, we performed non-covalent docking with the Glide program using the same workflow used for non-covalent redocking.

### Results

Among the molecules with the best docking scores, obtained from both covalent and non-covalent docking of the 300 initial set of compounds, a total of 30 compounds were short-listed after according to their in silico scores. The selected compounds were the following: Cynarin, Rupintrivir, Eravaciclyine dihydrochloride, Telaprevir, Cobiscitat, Bitolterol mesylate, Olcegepant, Epirubicin hydrochloride, Voruciclib hydrochloride, Azimilide hydrochloride, Florifenine, Diquafosol tetrasodium, Udenafil, Tafenoquine succinate, Darapladib, Lapatinib ditosylate, Oprozomib, Zaragozic acid A trisodium, Poldine methylsulfate, Pipequaline, Lasmiditan hydrochloride, Ractopamine hydrochloride, Tozasertib, Etafenone hydrochloride, Rimcazole dihydrochloride, Pibenzimol, Salmeterol, Prexasertib dihydrochlorided, Abexinostat and Danusertib.

Further experimental work was carried out with the above mentioned 30 compounds as explained below.

### Example 3

### Measurement of SARS-CoV-2 3CLpro Activity Using FRET Assay.

An intramolecularly quenched fluorogenic substrate containing a donor and an acceptor chromophore in the same molecule can be used to provide a rapid simple method of measuring enzyme activity and the availability of a fluorescence resonance energy transfer (FRET) synthetic peptide allows the use of high-throughput screening to identify lead candidates in compound libraries.

EDANS (5-((2-Aminoethyl)amino)naphthalene-1-sulfonic acid) is a donor for FRET-based protease substrates. EDANS is often paired with the acceptor Dabcyl (4-(4-dimethylaminophenyl)-diazenylbenzoic acid). The combination can be used in enzyme assays. When the two compounds are in close proximity, most of the energy emitted from EDANS will be quenched by DABCYL. However, if the compounds are separated (for example, by substrate cleavage) EDANS will fluoresce, giving an indication of enzyme presence.

The custom-synthesized fluorogenic substrate, DABCYL-KTSAVLQSGFRKME-EDANS (ANYGEN, Gwangju, Korea) [DABCYL-SEQ ID NO: 1-EDANS], was used as a substrate for the proteolytic assay using the SARS-CoV 3CLpro. This substrate contains the nsp4/nsp5 cleavage sequence GVLQ ↓ SG and works as a generic peptide substrate for many coronavirus including the SARS-CoV-2 3CLpro.

The peptide was dissolved in distilled water and incubated with the protease. A SpectraMax i3x Multi-mode microplate reader (Molecular Devices) was used to measure spectral-based fluorescence. The proteolytic activity was determined at 37° C. by following the increase in fluorescence (λexcitation 340 nm, ,emission 490 nm, bandwidths 9, 15 nm, respectively) of EDANS upon peptide hydrolysis as a function of time. Assays were conducted in black, 96-well plates (Nunc) in 300 μl assay buffers containing protease and substrate as follow; For the SARS-CoV-2 3CLpro assay, 2.04 μl of 0.294 mM protease containing 20 mM Tris pH 7.5 was incubated with 7.5 μl of 0.1 mM substrate at 37° C. for 2 h 30 minutes before measuring Relative Fluorescence Unit (RFU).

Before the assay, the emission spectra of the compounds to be tested were surveyed after illuminating at 340 nm to avoid the overlapping with the emission spectrum of EDANS. Every compound was suitable to be tested. The final concentration of the protease was 2 μM, the peptide was at 2.5 μM and the test compound was used at a concentration range of 0.5 μM˜20 μM. At first, the SARS-CoV-2 3CLpro and test compound were mixed and pre-incubated at room temperature for 1 h.

The reaction was initiated by the addition of the substrate and each well was incubated at 37° C. for 2 hours 30 minutes. After that, we measured the fluorescence of the mixture on the black 96-well plate using the endpoint mode of SpectraMax i3x where the excitation wavelength was fixed to 340 nm and the emission wavelength was set to 490 nm using 9, 15 nm bandwidth, respectively. All reactions were carried out in triplicate. The IC50 value of the test compounds, which is the value causing 50% inhibition of the catalytic activity of the SARS-CoV-2 3CLpro, was calculated by nonlinear regression analysis using GraphPad Prism 7.03 (GraphPad Software, San Diego, Calif., USA).

### Test Compounds

The 30 compounds of example 2 were tested according the assay described above.

### Results

Table 1 summarizes the results of covalent docking scores and IC₅₀ for the 30 selected compounds tested in both examples 2 and 3 (na: not active):

TABLE 1

| | Docking score | |
|---|---|---|
| Compound name | (Covalent) | IC₅₀ |
| CYNARIN | −7,020 (−7,747) | 1.815 μM |
| RUPINTRIVIR | −8,557 (−6,510) | na |
| ERAVACYCLINE diHcl | −6,458 (−6,090) | 1.645 μM |
| TELAPREVIR | −8,854 | na |
| COBICISTAT | −8,656 | na |
| BITOLTEROL mesylate | −8,028 | na |
| OLCEGEPANT | −7,832 | na |
| EPIRUBICIN HCl | −7,826 | na |
| VORUCICLIB HCl | −7,793 | na |
| AZIMILIDE diHcl | −7,642 | na |
| FLORIFENINE | −7,597 | na |
| DIQUAFOSOL | −7,551 | na |
| TETRASODIUM | | |
| UDENAFIL | −7,415 | na |
| TAFENOQUINE succinate | −7,362 | na |
| DARAPLADIB | −7,295 | na |
| LAPATINIB DITOSYLATE | −7,234 | na |
| OPROZOMIB | −7,223 | na |
| ZARAGOZIC ACID A | −7,159 | na |
| trisodium | | |
| POLDINE | −7,124 | na |
| METHYLSULFATE | | |
| PIPEQUALINE | −7,081 | na |
| LASMIDITAN HCl | −6,896 | na |
| RACTOPAMINE HCl | −6,878 | na |
| TOZASERTIB | −7,208 | na |
| ETAFENONE HCl | −6,917 | na |
| RIMCAZOLE diHCl | −6,876 | na |
| PIBENZIMOL | −5,977 | na |
| SALMETEROL | −6,797 | na |
| PREXASERTIB diHCl | −6,558 | 1.996 μM |
| ABEXINOSTAT | −6,161 | na |
| DANUSERTIB | −5,734 | na |

It is very striking that most of the compounds that had a good docking score in example 2 showed no inhibition activity when tested against SARS-CoV-2 3CLpro. Actually only 3 out of the 30 tested compounds were active in vitro: eravacycline, cynarin and prexasertib. Cynarin and eravacycline had a high docking score and were also predicted to form a covalent bond with the protease. Rupintrivir, however, had a similarly high docking score and was also predicted to form a covalent bond, but it was not active in vitro, like many more compounds that appeared in the list with high docking scores.

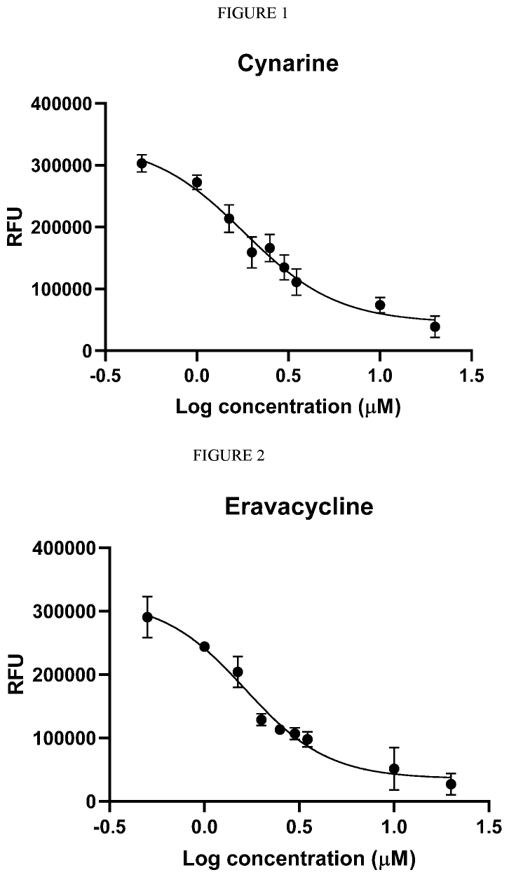

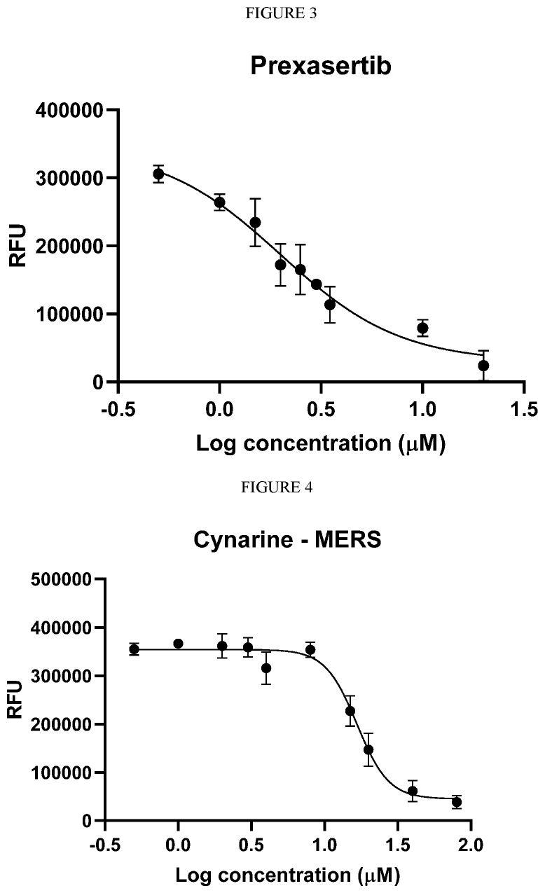

Cynarine, eravacycline, and prexasertib showed a prominent inhibitory activity against SARS-CoV-2 3CLpro. The dose response curves were plotted as log inhibitor concentration versus percent fluorescence inhibition (FIG. 1 for Cynarine, FIG. 2 for Eravacycline and FIG. 3 for Prexasertib). Cynarine, eravacycline, and prexasertib presented a strong inhibitory activity with IC50 values of 1.815 μM, 1.645 μM and 1.996 μM respectively.

### MERS-CoV

### Example 4

### Protein Expression and Purification of the MERS 3CL Protease

The coding sequence of MERS-CoV nsp5, a 3C-like protease (NCBI Ref. seq. YP_009047217.1 dated 13 Aug. 2018) was synthesized chemically by Bioneer and cloned into a bacteriophage T7-based expression vector. The plasmid DNA was transformed into E. coli BL21 (DE3) for protein expression. E. coli BL21 (DE3) cells were grown on Luria-Bertani (LB) agar plates containing 150 μg/ml ampicillin. Several colonies were picked and grown in capped test-tubes with 10 ml LB broth containing 150 μg/ml ampicillin. A cell stock composed of 0.85 ml culture and 0.15 ml glycerol was prepared and frozen at −80° C. for use in a large culture.

The frozen cell stock was grown in 5 ml LB medium and diluted into 2,000 ml fresh LB medium. The culture was incubated at 37° C. with shaking until an OD600 of 0.6-0.8 was reached. At this point, expression of MERS-CoV 3CLpro was induced using isopropyl-β-d-1-thiogalactopyranoside (IPTG) at a final concentration of 1 mM. The culture was further grown at 37° C. for 3 hr in a shaking incubator.

Cells were harvested by centrifugation at 7,650 g (6,500 rev min-1) for 10 min in a high-speed refrigerated centrifuge at 4° C. The cultured cell paste was resuspended in 25 ml of a buffer consisting of 50 mM Tris-HCl pH 8.0, 100 mM NaCl, 10 mM imidazole, 1 mM phenylmethylsulfonyl fluoride (PMSF) and 10 μg/ml DNase I. The cell suspension was disrupted using an ultrasonic cell disruptor (Digital Sonifier 450; Branson). Cell debris was pelleted by centrifugation at 24,900 g (15,000 rev min-1) for 30 min in a high-speed refrigerated ultra-centrifuge at 4° C.

The protein was purified by affinity chromatography using a 5 ml Hi-Trap Q column (GE Healthcare) followed by a 5 ml Hi-Trap Blue column (GE Healthcare).

### Example 5

### Measurement of MERS-CoV 3CLpro Activity Using FRET Assay.

The custom-synthesized fluorogenic substrate, DABCYL-KTSAVLQSGFRKME-EDANS (ANYGEN) [DABCYL-SEQ ID NO: 1-EDANS], was used as a substrate for the proteolytic assay using MERS-CoV 3CLpro. This substrate contains the nsp4/nsp5 cleavage sequence, GVLQ↓SG, and works as a generic peptide substrate for many coronavirus including MERS-CoV 3CLpro.

The peptide was dissolved in distilled water and incubated with each protease. A SpectraMax i3x Multi-mode microplate reader (Molecular Devices) was used to measure spectral-based fluorescence. The proteolytic activity was determined at 37° C. by following the increase in fluorescence (λexcitation=340 nm, λemission=490 nm, and widths=9, 15 nm, respectively) of EDANS upon peptide hydrolysis as a function of time. Assays were conducted in black, 96-well plates (Nunc) in 350 μl assay buffers containing protease and substrate as follows: for the MERS-CoV 3CLpro assay, 1.84 μl of 0.19 mM protease containing 20 mM Tris pH 8.0 was incubated with 8.75 μl of 0.1 mM substrate at 37° C. for 2 hr before measuring Relative Fluorescence Unit (RFU).

Before the assay, the emission spectra of test compounds were surveyed after illuminating at 340 nm to avoid the overlapping with the emission spectrum of EDANS. Every compound was suitable to be tested. The final concentration of the protease was 1 μM, the peptide was at 2.5 μM and test compound was used at a concentration range of 0.5 μM-80 μM. At first, MERS-CoV 3CLpro and chemical were mixed and preincubated at room temperature for 1 hr.

The reaction was initiated by the addition of the substrate, and each well was incubated at 37° C. for 2 hr. After 2 hr, the fluorescence of the mixture was measured on the black 96-well plate using the end-point mode of SpectraMax i3x where the excitation wavelength was fixed to 340 nm and the emission wavelength was set to 490 nm using 9, 15 nm bandwidth, respectively. All reactions were carried out in triplicate. IC50 value which is the value causing 50% inhibition of catalytic activity of MERS-CoV 3CLpro was calculated by non-linear regression analysis using graphpad prism 7.03 (GraphPad Software).

### Results

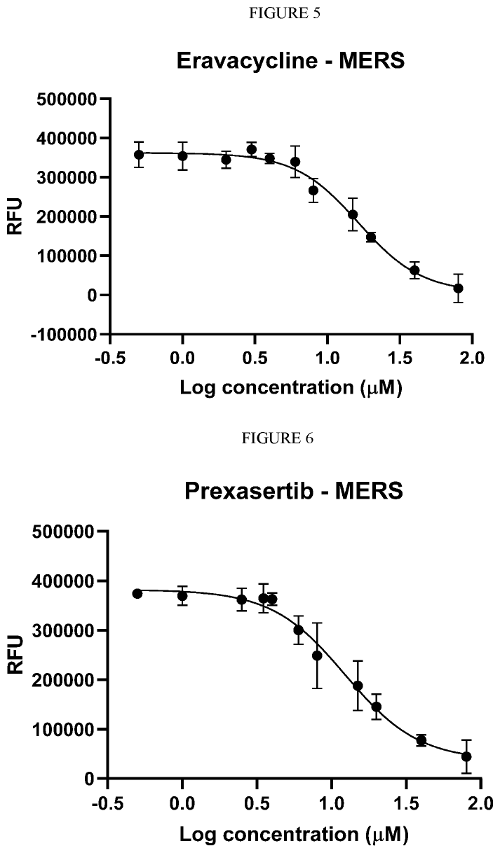

Cynarine, eravacycline, and prexasertib showed a prominent inhibitory activity against MERS-CoV-2 3CLpro. The dose response curves of inhibition of MERS 3CL protease were plotted as log inhibitor concentration versus percent fluorescence inhibition (FIG. 4 for Cynarine, FIG. 5 for eravacycline, and FIG. 6 for prexasertib). Cynarine, eravacycline, and prexasertib presented a strong inhibitory activity with IC50 values of 16.67 μM, 16.36 μM and 12.38 μM respectively.

The results show that these compounds are able to inhibit the enzyme SARS-CoV-2 and MERS-CoV 3CL proteases making them suitable candidates for reducing the rate of replication of respiratory syndrome-related coronavirus selected from the species “Middle East respiratory syndrome-related coronavirus” (such as MERS-CoV) and “Severe respiratory syndrome-related coronavirus” (such as SARS-CoV and SARS-CoV-2).

### Example 6

### SARS-CoV-2 Infection Assays in VeroE6 Cells

### Methods:

A 384 tissue culture plate was inoculated with 1.2×10⁴ veroE6 cells per well. Twenty-four hours later, DMSO was used to prepare serial dilutions of the compounds (10 points, with 50 μM or 150 μM as the highest concentration) and added to cells in two sets of duplicates. About an hour after the compound treatment at the BSL3 facility, one set of vero cells were infected with SARS-CoV-2 at MOI (multiplicity of infection) of 0.0125 and cultured at 37 C for 24 hours. The other set of vero cells treated with compounds were left un-infected to monitor the cytotoxic activity of the compounds. After fixing the cells with 4% paraformaldehyde (PFA), permeabilization was performed. The cells were processed with the anti-SARS-CoV-2 Nucleocapsid (N) antibody as the primary antibody and then treated with Alexa-Fluor-488-conjugated goat anti-rabbit IgG as the secondary antibody. Cell nuclei were stained with Hoechst 33342. Fluorescence images were captured using a large-capacity image analysis device (Opera, Perkin Elmer). The acquired images were analyzed using the Image Mining (IM) software, an in-house analysis program. The total number of the cells per well was obtained by counting the number of nuclei stained with Hoechst 33342. The infected cells were counted with the number of cells expressing nucleocapsid protein. The infection ratio was calculated with the ratio of cells expressing nucleocapsid protein to the total number of cells. The infection rate was normalized by setting the average infection rate of wells including uninfected cells (mock) as 0% and wells including infected cells (0.5% DMSO group) without compounds as 100%. The response curves according to the concentration of compounds together with IC₅₀ and CC₅₀ values were obtained using the Y=Bottom+(Top Bottom)/(1+(IC₅₀/X)Hillslope) formula running the XLFit 4 (IDBS) software. All the IC₅₀ and CC₅₀ values were measured by two replicates and the reliability of the assays was judged with Z′-factor and the % coefficient of variation (% CV).

The following compounds were tested following the above-mentioned: chloroquine, remdesivir and eravacycline.

### Results and Discussion

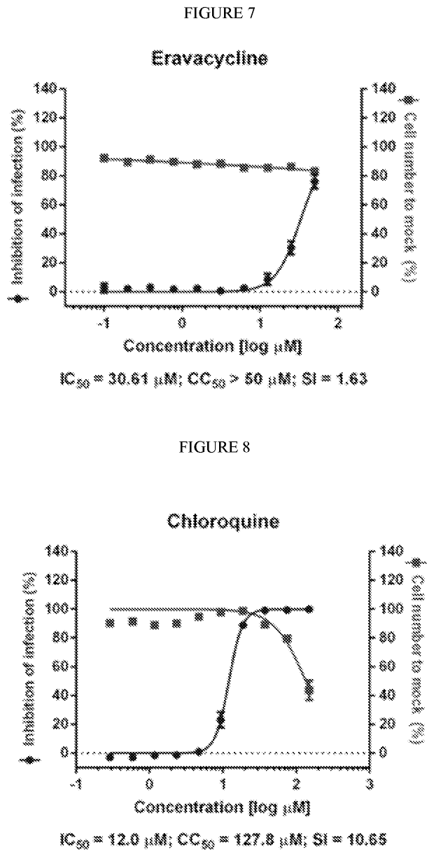

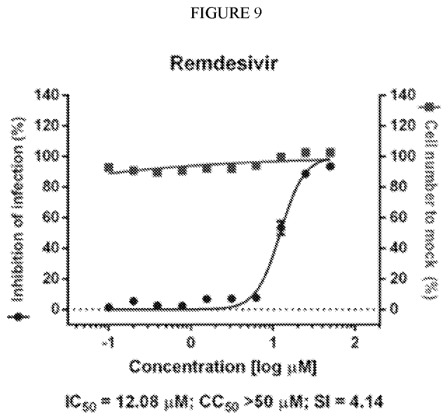

The 3CLprotease is essential for the life cycle of coronaviruses in infected cells, as it mediates the release of the viral proteins that make the machinery for viral transcription, replication and assembly of new viruses. The antiviral activities of eravacycline, chloroquine and remdesivir were tested in SARS-CoV-2 infected VeroE6 cells. Cells were treated with different concentrations of test compounds for one hour prior to the infection with SARS-CoV-2 at a multiplicity of infection of 0.0125. Cell infection was measured 24 h after by viral nucleocapsid protein N staining, and the cytotoxicity of the compounds was also tested in parallel in the absence of virus. As shown in FIG. 7, Eravacycline inhibited cell infection with an IC₅₀ of 30.61 μM without affecting cell viability. In comparison, remdesivir and chloroquine inhibited infection with IC₅₀ of approximately 12 μM, with significant cytotoxicity observed in cells treated with chloroquine at high concentrations.

1.-22. (canceled)

23. A method for the treatment and/or prevention of viral infections by betacoronaviruses comprising the administration to a patient in need thereof of a compound selected from the group consisting of eravacycline, cynarine and prexasertib or a pharmaceutically acceptable salt thereof.

24. The method according to claim 22 wherein the betacoronaviruses are selected from respiratory syndrome-related coronaviruses selected from the species “Severe respiratory syndrome-related coronavirus” and “Middle East respiratory syndrome-related coronavirus”.

25. The method according to claim 23, wherein the betacoronaviruses are respiratory syndrome-related coronaviruses selected from the group consisting of SARS-CoV-2, SARS-CoV and MERS-CoV.

26. (canceled)

27. The method according to claim 23, wherein the viral infection is by SARS-CoV-2 virus.

28. A method for the treatment and/or prevention of viral infections by betacoronaviruses, in particular by respiratory syndrome-related coronavirus selected from the species “Middle East respiratory syndrome-related coronavirus” (MERS-CoV) and “Severe respiratory syndrome-related coronavirus” (SARS-CoV and SARS-CoV-2), comprising the administration to a patient in need thereof of a combination comprising a compound selected from the group consisting of cynarine, eravacycline and prexasertib or a pharmaceutically acceptable salt thereof and one or more additional compounds selected from the group consisting of cynarine, eravacycline, prexasertib, remdesivir, ribavirin, chloroquine, hydroxychloroquine, oseltamivir, favipiravir, lopinavir, ritonavir, umifenovir, darunavir, cobicistat, galidesivir, danoprevir, ASC09, baloxavir marboxil, azvudine, vicromax, cidofovir, pleconaril, valacyclovir, abacavir, dolutegravir, lamivudine, emtricitabine, tenofovir, ledipasvir, sofosbuvir, velpatasvir, pimodivir, galidesivir, acyclovir, brivudine, famciclovir, penciclovir, ganciclovir, amantadine, rimantadine, nelfinavir, grazoprevir, atazanavir, voxilaprevir, saquinavir, amprenavir, indinavir, asunaprevir, fosamprenavir, danoprevir, simeprevir, vaniprevir, narlaprevir, boceprevir, tipranavir, glecaprevir, zanamivir, teicoplanin, oritavancin, dalbavancin, monensin, mycophenolic acid, gemcitabine, navitoclax, luteolin, glycyrrhizin, eflornithine, silvestrol, emodin, amiodarone, homoharringtonine, alisporivir, hexachlorophene, camostat, memantine, indomethacin, telavancin, trametinib, N3, ebselen, disulfiram, carmofur, shikonin, EIDD-1931, EIDD-2801, ivermectin, tideglusib, PX-12, hydrocortisone, methylprednisolone, dexamethasone, ciclesonide, naproxen, peginterferon alpha2b, colchicine, opaganib, Novaferon, SNG001, peginterferon lambda, Rebif, sargramostim, anakinra, baricitinib, antibodies from recovered COVID19 patients, bevacizumab, PD-1 antibody, leronlimab, camrelizumab, sarilumab, tocilizumab, gimsilumab, TJM2, lenzilumab, siltuximab, eculizumab, mavrilimumab, canakinumab, namilumab, emapalumab, meplazumab, ruxolitinib, fingolimod, camostat mesylate, ifenprodil, APN01, brilacidin, BXT-25, nafamostat, losartan, alteplase, AT001, plitidepsin, dipyridamole, ebastine, azithromycin, solnatide, selinexor or a pharmaceutically acceptable salt thereof.

29. The method according to claim 28, wherein the combination comprises eravacycline or a pharmaceutically acceptable salt thereof.

30. The method according to claim 28, wherein the viral infection is by SARS-CoV-2 virus.

31. The method according to claim 28, wherein the combination is contained in a composition in the form of a dry powder.

32. The method according to claim 31, wherein the composition is contained in capsules or cartridges for use in an inhalation device.

33. The method according to claim 28, wherein the combination comprises eravacycline dihydrochloride.


SEQUENCE LISTING (1 sequence)

SEQ ID NO: 1 - protein, 14 aa, Artificial Sequence
  Substrate for SARS-CoV-2 3CLpro
KTSAVLQSGFRKME